(19) United States
(12) Patent Application Publication
(10) Pub. No.: US 2024/0350565 A1
(43) Pub. Date: Oct. 24, 2024

(54) POSTBIOTIC

(71) Applicant: SOCIETE DES PRODUITS NESTLE S.A., Vevey (CH)

(72) Inventors: KATJA JOHNSON, Konolfingen (CH); JALIL BENYACOUB, Epalinges (CH)

(21) Appl. No.: 18/683,538

(22) PCT Filed: Aug. 18, 2022
(86) PCT No.: PCT/EP2022/073085

§ 371 Date: Feb. 14, 2024

(30) Foreign Application Priority Data
Aug. 19, 2021 (EP) 21192227.3

(51) Int. Cl. A61K 35/745; A61K 9/16; C12N 1/20

(52) U.S. Cl. CPC A61K 35/745; A61K 9/16; C12N 1/20

(57) ABSTRACT

The present invention provides use of a Bifidobacterium lactis supernatant as a bifidogenic factor. The present invention also provides a Bifidobacterium lactis supernatant for use in enhancing the growth of bifidobacteria in the gastrointestinal tract of a subject. The present invention also provides a Bifidobacterium lactis supernatant for use in treating or preventing a gastrointestinal disease by enhancing the growth of bifidobacteria in the gastrointestinal tract of a subject.

### FIELD OF THE INVENTION

The present invention relates to postbiotics and their use as bifidogenic factors. The present invention also relates to the use of postbiotics in treating or preventing a gastrointestinal disease by enhancing thegrowth of bifidobacteria in the gastrointestinal tract of a subject.

### BACKGROUND TO THE INVENTION

Bifidobacteria are one of the major genera of bacteria that make up the gastrointestinal tract microbiota in mammals. Bifidobacteria are among the first microbial colonizers of the intestines of newborns, and play key roles in the development of their physiology (Hidalgo-Cantabrana, C., et al., 2017. Microbiology spectrum, 5 (3), pp. 5-3). Moreover, alterations in composition and function of bifidobacteria have been associated with several gastrointestinal diseases, including inflammatory bowel disease, colorectal cancer, and irritable bowel syndrome (Tojo, R., et al., 2014. World journal of gastroenterology: WJG, 20 (41), p. 15163).

The use of probiotics, including bifidobacteria strains, in preventive medicine to maintain a healthy intestinal function is well documented and have been proposed as therapeutic agents for gastrointestinal disorders. Numerous studies have demonstrated the capability of prebiotics (e.g. inulin, arabinoxylans, galactooligosaccharides, and fructooligosaccharides) to promote bifidobacterial presence within the microbiota (Hidalgo-Cantabrana, C., et al., 2017. Microbiology spectrum, 5 (3), pp. 5-3).

However, existing solutions to enhancing the growth of bifidobacteria have a number of drawbacks. Probiotics need to be kept alive, limiting their application. Prebiotics generally require a dose of a few grams to deliver a benefit, which can make them less suitable for supplements since the capsule size typically limits ingredient dosage to below the gram range.

Thus, there is a demand for alternative agents and compositions which can enhance the growth of bifidobacteria in the gastrointestinal tract of a subject.

### SUMMARY OF THE INVENTION

Bifidobacterium animalis subsp. lactis (also known as Bifidobacterium lactis or B. lactis) supernatant has surprisingly been found to enhance growth of bifidobacteria, indicating a surprising bifidogenic effect. The supernatant of Bifidobacterium lactis was surprisingly found to enhance growth of bifidobacteria to a significantly greater extent than supernatant of Lactobacillus rhamnosus.

The bifidogenic effect of the pure liquid supernatant of Bifidobacterium lactis was advantageously maintained when turning the supernatant into a powder. A combination of a Bifidobacterium lactis with the Bifidobacterium lactis supernatant powder also showed a significant bifidogenic effect. The powder form is stable for storage and is suitable for supplements.

In one aspect, the present invention provides a Bifidobacterium lactis supernatant.

In another aspect, the present invention provides use of a Bifidobacterium lactis supernatant as a bifidogenic factor.

In another aspect, the present invention provides a Bifidobacterium lactis supernatant for use in enhancing the growth of bifidobacteria in the gastrointestinal tract of a subject.

In another aspect, the present invention provides a Bifidobacterium lactis supernatant for use in treating or preventing a gastrointestinal disease by enhancing the growth of bifidobacteria in the gastrointestinal tract of a subject.

Any suitable Bifidobacterium lactis strain may be used in the present invention. In some embodiments, the Bifidobacterium lactis is selected from: Bifidobacterium animalis subsp. lactis CNCM I-3446, Bifidobacterium animalis subsp. lactis BI12, Bifidobacterium animalissubsp. lactis BLC1, Bifidobacterium animalis subsp. lactis DSM10140, Bifidobacterium animalis subsp. lactis V9, Bifidobacterium animalis subsp. lactis BI-04, Bifidobacterium animalis subsp. lactis Bi-07, Bifidobacterium animalis subsp. lactis B420, Bifidobacterium animalis subsp. lactis BB-12, Bifidobacterium animalis subsp. lactis AD011, Bifidobacterium animalis subsp. lactis HN019, Bifidobacterium animalis subsp. lactis DN-173 010, Bifidobacterium animalis subsp. lactis ATCC 27536, and Bifidobacterium animalis subsp. lactis VTT E-012010. In some embodiments, the Bifidobacterium lactis is a Bifidobacterium animalis subsp. lactis having at least 99.0%, at least 99.1%, at least 99.2%, at least 99.3%, at least 99.4%, at least 99.5%, at least 99.6%, at least 99.7%, at least 99.8%, or at least 99.9% sequence identity to Bifidobacterium animalis subsp. lactis CNCM I-3446. In some embodiments, the Bifidobacterium lactis is Bifidobacterium animalis subsp. lactis CNCM I-3446.

The Bifidobacterium lactis supernatant may be obtained or obtainable by culturing Bifidobacterium lactis in a suitable culture media. In some embodiments, the culture medium comprises sugar and yeast extract, and, optionally, sodium ascorbate and/or polysorbate. In some embodiments, the culture media comprises: (i) about 1 wt % to about 6 wt %, or about 2 wt % to about 4 wt % sugar; (ii) about 1 wt % to about 10 wt %, or about 1 wt % to about 6 wt %, or about 2 wt % to about 4 wt % yeast extract; (iii) about 0 wt % to about 0.5 wt %, or about 0.1 wt % to about 0.2 wt % sodium ascorbate; and/or (iv) about 0 wt % to about 1 wt %, or about 0 wt % to about 0.3 wt % polysorbate. In some embodiments, the sugar is glucose, dextrose and/or glucose syrup. In some embodiments, the pH is controlled at a pH of from about 5 to about 7, a pH of from about 5.5 to about 6.5, or a pH of about 6. The Bifidobacterium lactis may be cultured under any suitable conditions. In some embodiments, the Bifidobacterium lactis is cultured until stationary phase is reached. In some embodiments, the Bifidobacterium lactis is cultured under anaerobic conditions.

Any suitable processing steps may be used to obtain a Bifidobacterium lactis supernatant. The Bifidobacterium lactis supernatant may be obtained or obtainable by removing all or substantially all the Bifidobacterium lactis cells from a Bifidobacterium lactis fermentate. In some embodiments, the Bifidobacterium lactis cells are removed by centrifugation.

In some embodiments, the Bifidobacterium lactis supernatant is pasteurised. In some embodiments, the Bifidobacterium lactis supernatant is dried. In some embodiments, the Bifidobacterium lactis supernatant is dried by spray-drying. In some embodiments, the Bifidobacterium lactis supernatant is spray-dried with a carrier material selected from one or more of: oat fibre, maltodextrin, acacia gum, starch and inulin. In some embodiments, the Bifidobacterium lactis supernatant is spray-dried with acacia gum. In some embodiments, the Bifidobacterium lactis supernatant and carrier material are mixed in a total solids ratio of from about 1:3 to about 2:1 (carrier: supernatant dry solids), preferably wherein the total solids ratio is about 1:1 (carrier: supernatant dry solids).

The Bifidobacterium lactis supernatant may be provided in any suitable form. In some embodiments, the Bifidobacterium lactis supernatant is in a form suitable for oral administration. In some embodiments, the Bifidobacterium lactis supernatant is in the form of a supplement or a nutritional composition. In some embodiments, the Bifidobacterium lactis supernatant is in the form of a capsule or a tablet.

During culturing the Bifidobacterium lactis will consume nutrients in the culture media (e.g. sugar and amino acids) and enrich the culture media with various soluble factors (e.g. organic acids and other metabolites). In some embodiments, compared to Bifidobacterium lactis culture media the Bifidobacterium lactis supernatant has: (i) a decreased concentration of total sugar; (ii) an increased concentration of total acids; and/or (iii) a decreased concentration of total amino acids. In some embodiments, compared to Bifidobacterium lactis culture media: (i) the concentration of total sugar has decreased by at least about 50%, at least about 60%, at least about 70%, at least about 80%, or at least about 90%; (ii) the concentration of total acids has increased by at least about 70%, by at least about 80%, or by at least about 90% of the concentration decrease in total sugars; and/or (iii) the concentration of total amino acids in the media has decreased by at least about 0.1 wt %, by at least about 0.2 wt %, or by at least about 0.3 wt %.

In some embodiments, before pasteurisation the Bifidobacterium lactis supernatant has a viable cell count of from about 1×10⁷ to about 1×10⁹ cfu/ml. In some embodiments, the Bifidobacterium lactis supernatant comprises: (i) about 4 wt % or less, about 3 wt % or less, about 2 wt % or less, about 1 wt % or less, or about 0.5 wt % or less total sugars; (ii) about 0.5 wt % or more, about 1 wt % or more, about 1.5 wt % or more, or about 2 wt % or more total acids; and/or (iii) about 3.5 wt % or less, about 2 wt % or less, about 1 wt % or less, about 0.8 wt % or less, or about 0.6 wt % or less total amino acids. In some embodiments, the Bifidobacterium lactis supernatant has a pH of from about 5 to about 7, from about 5.5. to about 6.5, or of about 6, preferably wherein the Bifidobacterium lactis supernatant has a pH of about 6.2.

The Bifidobacterium lactis supernatant may be used in combination with any other suitable agent or composition. In some embodiments, the Bifidobacterium lactis supernatant is used in combination with one or more probiotics, prebiotics, or synbiotics, preferably wherein the Bifidobacterium lactis supernatant is used in combination with one or more probiotics. In some embodiments, the Bifidobacterium lactis supernatant is used in combination with a Bifidobacterium lactis probiotic, preferably wherein the probiotic Bifidobacterium lactis is the same as the Bifidobacterium lactis from which the supernatant is derived.

The Bifidobacterium lactis supernatant may enhance the growth of one or more bifidobacterial phylogenetic groups selected from: the B. adolescentis group, the B. bifidum group, the B. longum group, and the B. pseudolongum group. The Bifidobacterium lactis supernatant may enhance the growth of one or more bifidobacterial species selected from: B. longum, B. animalis, B. adolescentis, B. bifidum, B. catenulatum, B. pseudocatenulatum, B. breve, B. pseudolongum, B. gallicum, B. angulatum, and B. faecale.

The Bifidobacterium lactis supernatant may be administered to any subject in need thereof. In some embodiments, the subject is human. In some embodiments, the subject has or is at risk of a gastrointestinal disease. In some embodiments, the subject has a low abundance of bifidobacteria in their gastrointestinal tract and/or faeces, preferably wherein the subject has a low abundance of bifidobacteria in their gastrointestinal tract and/or faeces relative to other microbiota.

The Bifidobacterium lactis supernatant may be used to treat or prevent disorders associated with decreased numbers of bifidobacteria in the gut, including gastrointestinal diseases, obesity, allergies, or regressive autism. The Bifidobacterium lactis supernatant may be used to treat or prevent any suitable gastrointestinal disease. In some embodiments, the gastrointestinal disease is a gastric disease or an intestinal disease, preferably an intestinal disease. In some embodiments, the gastrointestinal disease is selected from: antibiotic-associated diarrhea, Helicobacter pylori infection, an inflammatory bowel disease (IBD), irritable bowel syndrome (IBS), lactose intolerance, infectious diarrhea, colorectal cancer, chemotherapy-induced diarrhea, and necrotizing enterocolitis. In some embodiments, the gastrointestinal disease is selected from: antibiotic-associated diarrhea, Helicobacter pylori infection, an inflammatory bowel disease (IBD), and irritable bowel syndrome (IBS). In some embodiments, the IBD is Crohn's disease (CD), ulcerative colitis (UC), or pouchitis.

In another aspect, the present invention provides a supplement comprising a Bifidobacterium lactis supernatant. The supplement may be in the form of a capsule or a tablet.

In another aspect, the present invention provides a nutritional composition comprising a Bifidobacterium lactis supernatant.

The Bifidobacterium lactis supernatant present in the supplement or nutritional composition may be any Bifidobacterium lactis supernatant according to the present invention.

The Bifidobacterium lactis supernatant may be present in combination with any other suitable agent or composition. In some embodiments, the supplement or nutritional composition comprises one or more probiotics, prebiotics, or synbiotics, preferably wherein the supplement or nutritional composition comprises one or more probiotics. In some embodiments, the supplement or nutritional composition comprises a Bifidobacterium lactis probiotic, preferably wherein the probiotic Bifidobacterium lactis is the same as the Bifidobacterium lactis from which the supernatant is derived.

In another aspect, the present invention provides a method of manufacturing a Bifidobacterium lactis supernatant, the method comprising:
(a) culturing a Bifidobacterium lactis in a culture media to provide a Bifidobacterium lactis fermentate;
(b) removing substantially all the Bifidobacterium lactis cells from the Bifidobacterium lactis fermentate to provide a Bifidobacterium lactis supernatant; and
(c) optionally, pasteurising the Bifidobacterium lactis supernatant.

The method preferably further comprises a step (d) of drying the Bifidobacterium lactis supernatant, preferably wherein the Bifidobacterium lactis supernatant is dried by spray-drying.

The Bifidobacterium lactis supernatant manufactured by the method of the present invention may be any Bifidobacterium lactis supernatant according to the present invention.

In another aspect, the present invention provides a method of manufacturing a supplement comprising a Bifidobacterium lactis supernatant, the method comprising:
(a) culturing a Bifidobacterium lactis in a culture media to provide a Bifidobacterium lactis fermentate;
(b) removing substantially all the Bifidobacterium lactis cells from the Bifidobacterium lactis fermentate to provide a Bifidobacterium lactis supernatant;
(c) optionally, pasteurising the Bifidobacterium lactis supernatant;
(d) drying the Bifidobacterium lactis supernatant to provide a Bifidobacterium lactis supernatant powder; and
(e) encapsulating, compressing and/or packaging the Bifidobacterium lactis supernatant powder to provide a supplement comprising the Bifidobacterium lactis supernatant.

The steps may be carried out by any suitable means. In some embodiments, the Bifidobacterium lactis supernatant is dried by spray-drying. In some embodiments, the Bifidobacterium lactis supernatant powder is encapsulated to provide a capsule comprising the Bifidobacterium lactis supernatant.

The supplement manufactured by the method of the present invention may be any supplement according to the present invention.

### DESCRIPTION OF DRAWINGS

FIG. 1—Bifidogenic effect of Bifidobacterium lactis supernatant (absolute effect)

Absolute abundance of bifidobacteria at the start and after 24 h and 48 h in the blank incubation and in the incubations with heat treated B. lactis supernatant (SUP_BL) and heat treated L. rhamnosus supernatant (SUP_LR) using the gut microbial communities of three donors (A-C). Error bars indicate standard deviation of triplicates.

FIG. 2—Bifidogenic effect of Bifidobacterium lactis supernatant (change vs blank)

Difference between blank and treatments (heat treated B. lactis supernatant—SUP_BL; heat treated L. rhamnosus supernatant—SUP_LR) in log of absolute abundance of bifidobacteria at the start and after 24 h and 48 h using the gut microbial communities of three donors (Donors A-C). (A)—per donor. (B)—average of 3 donors. Error bars indicate standard deviation for 3 donors.

FIG. 3—Bifidogenic effect of Bifidobacterium lactis supernatant powder (absolute effect)

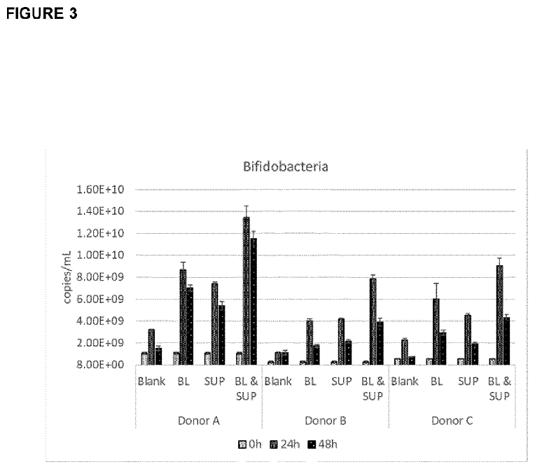

Absolute abundance of bifidobacteria at the start and after 24 h and 48 h in the blank incubation and in the incubations with B. lactis probiotic (BL), or heat treated B. lactis supernatant powder (SUP), or with a combination of B. lactis probiotic and heat treated B. lactis supernatant powder (BL & SUP) using the gut microbial communities of three donors (A-C). Error bars indicate standard deviation of triplicates.

FIG. 4—Bifidogenic effect of Bifidobacterium lactis supernatant powder (change vs blank)

Difference between blank and treatments (B. lactis probiotic—BL; heat treated B. lactissupernatant powder—SUP; combination of B. lactis probiotic and heat treated B. lactissupernatant powder—BL & SUP) in log of absolute abundance of bifidobacteria at the start and after 24 h and 48 h using the gut microbial communities of three donors (Donors A-C). (A)—per donor. (B)—average of 3 donors. Error bars indicate standard deviation for 3 donors.

### DETAILED DESCRIPTION

Various preferred features and embodiments of the present invention will now be described by way of non-limiting examples.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes”, “containing”, or “contains”, and are inclusive or open-ended and do not exclude additional, non-recited members, elements or steps. The terms “comprising”, “comprises” and “comprised of” also include the term “consisting of”.

Numeric ranges are inclusive of the numbers defining the range. As used herein the term “about” means approximately, in the region of, roughly, or around. When the term “about” is used in conjunction with a numerical value or range, it modifies that value or range by extending the boundaries above and below the numerical value(s) set forth. In general, the terms “about” and “approximately” are used herein to modify a numerical value(s) above and below the stated value(s) by 10%.

The publications discussed herein are provided solely for their disclosure prior to the filing date of the present application. Nothing herein is to be construed as an admission that such publications constitute prior art to the claims appended hereto.

This disclosure is not limited by the exemplary methods and materials disclosed herein, and any methods and materials similar or equivalent to those described herein can be used in the practice or testing of embodiments of this disclosure. The skilled person will understand that they can combine all features of the invention disclosed herein without departing from the scope of the invention as disclosed.

All publications mentioned in the specification are herein incorporated by reference.

### Bifidobacterium lactis Supernatant

In one aspect, the present invention provides a Bifidobacterium lactis supernatant.

As used herein, “supernatant” may refer to the spent or partially spent culture media in which cells have been cultured. Typically, all or substantially all the cells are removed from the culture media at the end of culturing. For example, a supernatant may be obtained or obtainable by a method comprising: (a) culturing cells in a culture media to provide a fermentate; and (b) removing all or substantially all the cells from the fermentate to provide a supernatant.

As used herein, a “Bifidobacterium lactis supernatant” may refer to a supernatant derived from a Bifidobacterium lactis culture. For example, a Bifidobacterium lactis supernatant may be obtained or obtainable by a method comprising: (a) culturing Bifidobacterium lactis in a culture media to provide a Bifidobacterium lactis fermentate; and (b) removing all or substantially all the Bifidobacterium lactis cells from the Bifidobacterium lactis fermentate to provide a Bifidobacterium lactis supernatant.

The Bifidobacterium lactis supernatant may be referred to as a postbiotic. As used herein, a “postbiotic” may refer to soluble factors (products or metabolic by-products), secreted by live bacteria, or released after bacterial lysis, such as enzymes, peptides, teichoic acids, peptidoglycan-derived muropeptides, polysaccharides, cell surface proteins, and organic acids (see e.g. Aguilar-Toalá, J. E., et al., 2018. Trends in Food Science & Technology, 75, pp. 105-114).

In one aspect, the present invention provides a postbiotic comprising or consisting of a Bifidobacterium lactis supernatant.

### Bifidobacterium lactis strains

Bifidobacterium lactis (also known as Bifidobacterium animalis subsp. lactis, NCBI: txid302911) is a gram-positive, anaerobic, rod-shaped bacterium of the Bifidobacterium genus which can be found in the large intestines of humans. Bifidobacterium animalis and Bifidobacterium lactis were previously described as two distinct species. Presently, both are considered B. animalis with the subspecies Bifidobacterium animalis subsp. animalis and Bifidobacterium animalis subsp. lactis (Masco, L., et al, 2004. International Journal of Systematic and Evolutionary Microbiology, 54 (4), pp. 1137-1143).

Any suitable Bifidobacterium lactis strain may be used in the present invention. For example, any Bifidobacterium lactis strain which is known to have a probiotic effect may be used in the present invention. Such Bifidobacterium lactis strains will be well known to the skilled person.

Suitably, the Bifidobacterium lactis may be selected from: Bifidobacterium animalis subsp. lactis CNCM I-3446, Bifidobacterium animalis subsp. lactis BI12, Bifidobacterium animalis subsp. lactis BLC1, Bifidobacterium animalis subsp. lactis DSM10140, Bifidobacterium animalis subsp. lactis V9, Bifidobacterium animalis subsp. lactis BI-04, Bifidobacterium animalis subsp. lactis Bi-07, Bifidobacterium animalis subsp. lactis B420, Bifidobacterium animalis subsp. lactis BB-12, Bifidobacterium animalis subsp. lactis AD011, Bifidobacterium animalis subsp. lactis HN019, Bifidobacterium animalis subsp. lactis DN-173 010, Bifidobacterium animalis subsp. lactis ATCC 27536, and Bifidobacterium animalis subsp. lactis VTT E-012010.

The Bifidobacterium lactis may be a Bifidobacterium animalis subsp. lactis having at least 99% sequence identity to any B. animalis subsp. lactis known to the skilled person. The Bifidobacterium lactis may be a Bifidobacterium animalis subsp. lactis having at least 99.0%, at least 99.1%, at least 99.2%, at least 99.3%, at least 99.4%, at least 99.5%, at least 99.6%, at least 99.7%, at least 99.8%, or at least 99.9% sequence identity to any B. animalis subsp. lactis known to the skilled person. There are at least 103 genome assemblies publicly available.

The Bifidobacterium lactis may be a Bifidobacterium animalis subsp. lactis having at least 99% sequence identity to Bifidobacterium animalis subsp. lactis CNCM I-3446, Bifidobacterium animalis subsp. lactis BI12 (Accession no. CP004053), Bifidobacterium animalis subsp. lactis BLC1 (Accession no. CP003039), Bifidobacterium animalis subsp. lactis DSM10140 (Accession no. CP001606), Bifidobacterium animalis subsp. lactis V9 (Accession no. CP001892), Bifidobacterium animalis subsp. lactis BI-04 (Accession no. CP001515), Bifidobacterium animalis subsp. lactis Bi-07 (Accession no. NC_017867), Bifidobacterium animalis subsp. lactis B420 (Accession no. NC_017866), Bifidobacterium animalis subsp. lactis BB-12 (Accession no. CP001853), Bifidobacterium animalis subsp. lactis AD011 (Accession no. CP001213), Bifidobacterium animalis subsp. lactis HN019 (Accession no. CP031154), Bifidobacterium animalis subsp. lactis DN-173 010, Bifidobacterium animalis subsp. lactis ATCC 27536, or Bifidobacterium animalis subsp. lactis VTT E-012010.

The Bifidobacterium lactis may be a Bifidobacterium animalis subsp. lactis having at least 99.0%, at least 99.1%, at least 99.2%, at least 99.3%, at least 99.4%, at least 99.5%, at least 99.6%, at least 99.7%, at least 99.8%, or at least 99.9% sequence identity to Bifidobacterium animalis subsp. lactis CNCM I-3446, Bifidobacterium animalis subsp. lactis BI12 (Accession no. CP004053), Bifidobacterium animalis subsp. lactis BLC1 (Accession no. CP003039), Bifidobacterium animalis subsp. lactis DSM10140 (Accession no. CP001606), Bifidobacterium animalis subsp. lactis V9 (Accession no. CP001892), Bifidobacterium animalis subsp. lactis BI-04 (Accession no. CP001515), Bifidobacterium animalis subsp. lactis Bi-07 (Accession no. NC_017867), Bifidobacterium animalis subsp. lactis B420 (Accession no. NC_017866), Bifidobacterium animalis subsp. lactis BB-12 (Accession no. CP001853), Bifidobacterium animalis subsp. lactis AD011 (Accession no. CP001213), Bifidobacterium animalis subsp. lactis HN019 (Accession no. CP031154), Bifidobacterium animalis subsp. lactis DN-173 010, Bifidobacterium animalis subsp. lactis ATCC 27536, or Bifidobacterium animalis subsp. lactis VTT E-012010.

Bifidobacterium animalis subsp. lactis CNCM I-3446, also named NCC 2818, was deposited with the Collection Nationale de Cultures de Microorganismes (CNCM), Institut Pasteur, 25 rue du Docteur Roux, F-75724 PARIS Cedex 15, France, on 7 Jun. 2005 and given the deposit number 1-3446.

In some embodiments, the Bifidobacterium lactis is a Bifidobacterium animalis subsp. lactis having at least 99% sequence identity to Bifidobacterium animalis subsp. lactis CNCM I-3446. In some embodiments, the Bifidobacterium lactis is a Bifidobacterium animalis subsp. lactis having at least 99.0%, at least 99.1%, at least 99.2%, at least 99.3%, at least 99.4%, at least 99.5%, at least 99.6%, at least 99.7%, at least 99.8%, or at least 99.9% sequence identity to Bifidobacterium animalis subsp. lactis CNCM I-3446.

In some embodiments, the Bifidobacterium lactis is Bifidobacterium animalis subsp. lactis CNCM I-3446.

### Supernatant Preparation

The Bifidobacterium lactis supernatant may be prepared by any suitable method. For example, preparation of bacterial supernatants is described in Moradi, M., et al., 2021. Enzyme and Microbial Technology, 143, p. 109722.

A Bifidobacterium lactis supernatant may be obtained or obtainable by a method comprising: (a) culturing a Bifidobacterium lactis in a culture media to provide a Bifidobacterium lactis fermentate; and (b) removing substantially all the Bifidobacterium lactis cells from the Bifidobacterium lactis fermentate.

Suitable culture conditions and processing steps will be well known to the skilled person. Exemplary culture conditions and processing steps are described herein.

### Culture Media

The Bifidobacterium lactis supernatant may be obtained or obtainable by culturing Bifidobacterium lactis in a culture media.

The Bifidobacterium lactis may be cultured in any suitable culture media. Suitable culture media will be well known to the skilled person. For example, Marsaux, B., et al., 2020. Nutrients, 12 (8), p. 2268 describe a suitable culture media for Bifidobacterium animalis subsp. lactis CNCM I-3446 made of dextrose at 2.8%, yeast-derived amino acids at 3%, and vitamin C.

Suitably, the culture media may be a commercially available media, such as MRS broth. MRS broth is non-selective medium for profuse growth of lactic acid bacteria and may comprise about 2% glucose, about 0.4% yeast extract, and about 0.1% polysorbate and may have a pH of about 6.2.

The culture media may comprise sugar, yeast extract, vitamin C, and/or polysorbate. Suitably, the culture media may comprise sugar and yeast extract. Suitably, the culture media may comprise sugar, yeast extract, and vitamin C. Suitably, the culture media may comprise sugar, yeast extract, vitamin C, and polysorbate.

Suitable sugars will be well known to the skilled person and include glucose, dextrose and/or glucose syrup. Suitably, the culture media may comprise from about 1 wt % to about 6 wt %, or from about 2 wt % to about 4 wt % sugar.

Yeast extract is the water-soluble portion of autolyzed yeast. Suitably, the culture media may comprise from about 1 wt % to about 10 wt %, from about 1 wt % to about 6 wt %, or from about 2 wt % to about 4 wt % yeast extract.

Suitable sources of vitamin C (also known as ascorbate or ascorbic acid) will be well known to the skilled person. For example, sodium ascorbate is one of a number of mineral salts of ascorbic acid. Suitably, the culture media may comprise from about 0 wt % to about 0.5 wt %, or from about 0.1 wt % to about 0.2 wt % sodium ascorbate.

Suitable polysorbates will be well known to the skilled person and include, for example, polysorbate 80. Suitably, the culture media may comprise from about 0 wt % to about 1 wt %, or from about 0 wt % to about 0.3 wt % polysorbate.

Suitably, the culture may comprise any other suitable components such as minerals (e.g. manganese sulphate) and/or peptone (e.g. yeast peptone).

In some embodiments, the culture media comprises from about 1 wt % to about 6 wt % sugar, from about 1 wt % to about 6 wt % yeast extract, from about 0 wt % to about 0.5 wt % sodium ascorbate, and from about 0 wt % to about 1 wt % polysorbate.

In some embodiments, the culture media comprises from about 2 wt % to about 4 wt % sugar, from about 2 wt % to about 4 wt % yeast extract, from about 0.1 wt % to about 0.2 wt % sodium ascorbate, and from about 0 wt % to about 0.3 wt % polysorbate.

Suitable pH will be well known to the skilled person and may be adjusted by any suitable means. Suitably, the culture media may have a pH of below about 7, such as from about 5.5 to about 6.5, or about 6.

### Culture Conditions

The Bifidobacterium lactis may be cultured under any suitable conditions. Suitable culture conditions will be well known to the skilled person. For example, Marsaux, B., et al., 2020. Nutrients, 12 (8), p. 2268 describe that Bifidobacterium animalis subsp. lactis CNCM I-3446 was incubated at 37° C.

Suitably, the Bifidobacterium lactis supernatant may be obtained or obtainable by culturing the Bifidobacterium lactis under anaerobic conditions.

Suitably, the Bifidobacterium lactis supernatant may be obtained or obtainable by culturing the Bifidobacterium lactis until the late log phase or stationary phase is reached. In some embodiments, the Bifidobacterium lactis supernatant is obtained or obtainable by culturing the Bifidobacterium lactis until the stationary phase is reached.

The stages recognised in cultivation of bacteria are known to the skilled person and include the “lag phase”, the “log phase”, the “stationary phase”, and the “death” phase. The “lag phase” is the phase in which there is no increase in the number of living bacterial cells. The “log phase” is the phase in which there is an exponential increase in the number of living bacterial cells. The “late log phase” may refer to the second half of the log phase, for example at least about 75%, at least about 80%, at least about 85%, at least about 90%, or at least about 95% through the log phase. The “stationary phase” is the phase in which the number of living bacterial cells remains constant. The “death phase” is the phase in which there is a decline in the number of living bacterial cells.

The culture phase may be determined using any suitable method known to the skilled person. For example, the beginning of the stationary phase may be determined as the time point when no additional acids are produced and base addition is no longer required to maintain the desired pH. Alternatively, the culture phase may be determined based on the optical density at 600 nm, which correlates with the bacterial concentration in a culture medium.

Suitably, the Bifidobacterium lactis supernatant may be obtained or obtainable by culturing at about 35° C. to about 40° C., or about 37° C. The temperature may be controlled by any suitable method known to the skilled person.

Suitably, the Bifidobacterium lactis supernatant may be obtained or obtainable by culturing with pH control. The pH may be controlled by any suitable method known to the skilled person, for example by using base addition. Suitably, the pH may be controlled at a pH of from about 5 to about 7, from about 5.5 to about 6.5, or about 6. Suitably, the pH may be controlled at a pH of 6.0.

Suitably, the Bifidobacterium lactis supernatant may be obtained or obtainable by culturing with stirring and/or with head space gassing. The stirring and/or head space gassing may be carried out by any suitable method known to the skilled person, for example by head space gassing with carbon dioxide.

Suitably, the Bifidobacterium lactis supernatant may be obtained or obtainable by culturing at about 37° C., with pH control at about pH 6, with stirring, and/or with head space gassing with carbon dioxide.

### Processing of Fermentate

The Bifidobacterium lactis supernatant may be obtained or obtainable by removing all or substantially all the Bifidobacterium lactis cells from a Bifidobacterium lactis fermentate.

The Bifidobacterium lactis cells may be removed by any suitable method known to the skilled person. For example, the Bifidobacterium lactis cells may be removed by centrifugation or filtration.

The Bifidobacterium lactis cells may be removed by centrifugation. Suitable centrifugation conditions will be well known to the skilled person, for example, centrifugation at from about 4000 to about 12000 g for about 10 min.

The Bifidobacterium lactis cells may be removed by filtration. Suitable filtration conditions will be well known to the skilled person, for example membrane filtration using a pore size of about 0.2 μm to remove all the Bifidobacterium lactis cells or a pore size of from about 0.3 μm to about 0.5 μm to remove substantially all the Bifidobacterium lactis cells.

As used herein, “substantially all” may mean the vast majority of Bifidobacterium lactis cells, e.g. at least 90%, at least 95%, or at least 99% of the Bifidobacterium lactis cells. Suitably, removing “substantially all” the Bifidobacterium lactis cells may mean that at least about 90%, at least about 95%, or at least about 99% of the Bifidobacterium lactis cells are removed.

In some embodiments, some Bifidobacterium lactis cells remain in the supernatant (e.g. following removal). For example, prior to inactivation, the Bifidobacterium lactis supernatant may have a viable cell count of from about 1×10⁶ to about 1×10¹⁰ cfu/ml or a viable cell count of from about 1×10⁷ to about 1×10⁹ cfu/ml.

In some embodiments, the remaining Bifidobacterium lactis cells are heat-inactivated. Suitable methods to heat-inactivate the Bifidobacterium lactis cells will be well known to the skilled person. For example, the Bifidobacterium lactis supernatant may be pasteurised. Suitable pasteurisation conditions will be well known to the skilled person. For example, the Bifidobacterium lactis supernatant may be pasteurised at a temperature of from about 70° C. to about 100° C. for about 10 to about 30 seconds, about 70° C. to about 100° C. for about 10 to about 15 seconds, or about 80° C. to about 100° C. for about 10 seconds.

In some other embodiments, the Bifidobacterium lactis supernatant may be a cell-free supernatant (i.e. 100% of the Bifidobacterium lactis cells are removed). A cell-free supernatant may be obtained by passing the supernatant through one or more filters to remove all the Bifidobacterium lactis cells. Suitable filtering conditions will be well known to the skilled person. For example, a 0.22 μm or a 0.4 μm pore size filter may be used to remove all Bifidobacterium lactis cells, or a 0.5 μm to 2.0 μm pore size filter may be used to remove substantially all Bifidobacterium lactis cells.

### Drying of Supernatant

Suitably, the Bifidobacterium lactis supernatant may be dried. Providing the Bifidobacterium lactis supernatant in a dry form may be more suitable for long-term storage. The Bifidobacterium lactis supernatant may be dried by any suitable method known to the skilled person. For example, the supernatant may be dried by spray-drying or lyophilisation. For the avoidance of doubt, the term “supernatant” includes a dried supernatant, e.g. a supernatant in a solid (e.g. powder) form).

In some embodiments, the Bifidobacterium lactis supernatant is spray-dried. Suitable methods for spray-drying a Bifidobacterium lactis supernatant will be well known to the skilled person (see e.g. Santos, D., et al., 2018. Biomaterials Physics and Chemistry-New Edition. InTech Open, Spray Drying: An Overview). For the avoidance of doubt, the term “supernatant” includes a spray-dried supernatant.

Suitably, the Bifidobacterium lactis supernatant may be spray-dried with a carrier material. Suitable carrier materials will be well known to the skilled person. For example, the carrier material may be selected from one or more of: oat fibre, maltodextrin, acacia gum, starch and inulin. In some embodiments, the carrier material is acacia gum.

Any suitable amount of carrier material may be used. For example, the Bifidobacterium lactis supernatant and carrier material may be mixed in a total solids ratio of from about 1:5 to about 5:1 (carrier: supernatant dry solids), or a total solids ratio of from about 1:3 to about 3:1 (carrier: supernatant dry solids), or a total solids ratio of from about 1:2 to about 2:1 (carrier: supernatant dry solids), or a total solids ratio of from about 1:1.5 to about 1.5:1 (carrier: supernatant dry solids), or a total solids ratio of from about 1:1 (carrier: supernatant dry solids).

### Form of Supernatant

The Bifidobacterium lactis supernatant may be provided in any suitable form. For example, the Bifidobacterium lactis supernatant can be in a solid (e.g. powder), liquid or gelatinous form. The Bifidobacterium lactis supernatant may be provided in a form suitable for oral or enteral administration. In some embodiments, the Bifidobacterium lactis supernatant is provided in a form suitable for oral administration.

In some embodiments, the Bifidobacterium lactis supernatant is provided in a solid (e.g. powder) form. Providing the Bifidobacterium lactis supernatant in the form of a solid (e.g. a powder) may be more suitable for consumption as a supplement (e.g. in tablet or capsule form).

The Bifidobacterium lactis supernatant may be provided in the form of a supplement or a nutritional composition.

In one aspect, the present invention provides a supplement comprising a Bifidobacterium lactis supernatant.

A “supplement” or “dietary supplement” may be used to complement the nutrition of an individual (it is typically used as such but it might also be added to any kind of compositions intended to be ingested). The supplement may be prepared in any suitable manner.

The supplement may be in the form of for example tablets, capsules, pastilles or a liquid. In some embodiments, the supplement is in the form of a capsule or a tablet.

The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents and gel forming agents. The supplement may also contain conventional pharmaceutical additives and adjuvants, excipients and diluents, including, but not limited to, water, gelatine of any origin, vegetable gums, lignin-sulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like. Further, the supplement may contain an organic or inorganic carrier material suitable for oral or parenteral administration as well as vitamins, minerals trace elements and other micronutrients in accordance with the recommendations of Government bodies such as the USRDA. The supplement may be provided in the form of unit doses.

In some embodiments, the supplement is a pet supplement. A “pet supplement” may refer to a supplement that is intended for pets. A pet may be an animal selected from dogs, cats, birds, fish, rodents such as mice, rats, and guinea pigs, rabbits, etc.

In one aspect, the present invention provides a nutritional composition comprising a Bifidobacterium lactis supernatant.

According to the present invention, a “nutritional composition” means a composition which nourishes a subject. The nutritional composition may be prepared in any suitable manner.

The nutritional composition is not particularly limited as long as it is suitable for administration (e.g. oral or intravenous administration). Examples of suitable nutritional compositions include foodstuffs, drinks, drug bases, and animal feeds.

The nutritional composition according to the invention may be an enteral nutritional composition. An “enteral nutritional composition” is a foodstuff that involves the gastrointestinal tract for its administration.

The nutritional composition may be suitable for infants. For example the nutritional composition may be an infant formula, a baby food, an infant cereal composition, or a fortifier. Suitably, the nutritional composition may be an infant formula or a fortifier.

Suitably, the nutritional composition may be a pharmaceutical composition. The form of the pharmaceutical preparation is not particularly limited, and examples include tablet, pill, powder, solution, suspension, emulsion, granule, capsule, syrup, and so forth. Additives widely used as pharmaceutical carriers for oral administration such as excipients, binders, disintegrating agents, lubricants, stabilizers, corrigents, diluents, and surfactants can be used. Examples of suitable binders include starch, gelatin, natural sugars such as glucose, anhydrous lactose, free-flow lactose, beta-lactose, corn sweeteners, natural and gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like.

In some embodiments, the nutritional composition is an animal feed, for example a pet food product (particularly a dry pet food product). A “pet food product” may refer to a nutritional product that is intended for consumption by pets. In some embodiments, the nutritional composition is a dog food product or a cat food product. In some embodiments, the nutritional composition is a veterinary composition.

### Supernatant Composition

As described above, the Bifidobacterium lactis supernatant may be obtained or obtainable by a method comprising (a) culturing a Bifidobacterium lactis in a culture media. During culturing the Bifidobacterium lactis will consume nutrients in the media (e.g. sugar and amino acids) and enrich the media with various soluble factors (e.g. organic acids and other metabolites).

The Bifidobacterium lactis -derived metabolites in a Bifidobacterium lactis supernatant may have diverse physicochemical and functional properties and can be analysed by any suitable method known to those of skill in the art. For example, suitable methods include gas chromatography, liquid chromatography, thin layer chromatography, spectrophotometric techniques, NMR spectroscopy, and FTIR spectroscopy (see e.g. Moradi, M., et al., 2021. Enzyme and Microbial Technology, 143, p. 109722).

Compared to Bifidobacterium lactis culture media the Bifidobacterium lactis fermentate or Bifidobacterium lactis supernatant may have: (i) a decreased concentration of total sugar; (ii) an increased concentration of total acids; and/or (iii) a decreased concentration of total amino acids.

As used herein, the term “total sugars” may refer to any sugar, including e.g. glucose and fructose. Suitably, compared to Bifidobacterium lactis culture media the concentration of total sugar in the Bifidobacterium lactis fermentate or Bifidobacterium lactis supernatant (e.g. prior to drying) may have decreased by at least about 50%, at least about 60%, at least about 70%, at least about 80%, at least about 90%, at least about 95%, or at least about 99%. Suitably, compared to Bifidobacterium lactis culture media the concentration of total sugar in the Bifidobacterium lactis fermentate or Bifidobacterium lactis supernatant (e.g. prior to drying) may have decreased by from about 50% to about 100%, or from about 80% to about 100%.

As used herein, the term “total acids” may refer to any organic acid (i.e. excluding amino acids), including e.g. acetic acid, lactic acid, and formic acid. Suitably, compared to Bifidobacterium lactis culture media the concentration of total acids in the Bifidobacterium lactis fermentate or Bifidobacterium lactis supernatant (e.g. prior to drying) may have increased by at least about 70%, by at least about 80%, or by at least about 90% of the concentration decrease in total sugars. For example, if the concentration of total sugars has decreased by 1 wt %, the concentration of total acids may have increased by at least about 0.7 wt %, at least about 0.8 wt %, or at least about 0.9 wt %. Suitably, compared to Bifidobacterium lactis culture media the concentration of total acids in the Bifidobacterium lactis fermentate or Bifidobacterium lactis supernatant (e.g. prior to drying) may have increased by from about 70% to about 90% of the concentration decrease in total sugars.

Suitably, compared to Bifidobacterium lactis culture media the concentration of total amino acids in the Bifidobacterium lactis fermentate or Bifidobacterium lactis supernatant (e.g. prior to drying) may have decreased by at least about 0.1 wt %, by at least about 0.2 wt %, by at least about 0.3 wt %, by at least 0.4 wt %, or by at least 0.5 wt %. Suitably, compared to Bifidobacterium lactis culture media the concentration of total amino acids in the Bifidobacterium lactis fermentate or Bifidobacterium lactis supernatant (e.g. prior to drying) may have decreased by from about 0.1 wt % to about 0.5 wt %.

Suitably, the Bifidobacterium lactis fermentate or Bifidobacterium lactis supernatant (e.g. prior to drying) may have a total solids content of from about 2 wt % to about 18 wt %, from about 2 wt % to about 10 wt %, from about 3 wt % to about 9 wt %, from about 4 wt % to about 8 wt %, from about 5 wt % to about 7 wt %, or about 6 wt %.

Suitably, the Bifidobacterium lactis fermentate or Bifidobacterium lactis supernatant (e.g. prior to drying) may comprise about 5 wt % or less, about 4 wt % or less, about 3 wt % or less, about 2 wt % or less, about 1 wt % or less, about 0.5 wt % or less, about 0.4 wt % or less, about 0.3 wt % or less, about 0.2 wt % or less, or about 0.1 wt % or less total sugars. In some embodiments, the supernatant may comprise less than 0.3 wt %, less than 0.2 wt %, or less than 0.1 wt % total sugars. Suitably, the Bifidobacterium lactis fermentate or Bifidobacterium lactis supernatant (e.g. prior to drying) may comprise about 0 wt % to about 5 wt %, about 0 wt % to about 2 wt %, about 0 wt % to about 1 wt %, about 0 wt % to about 0.5 wt %, or about 0 wt % to about 0.3 wt % total sugars.

Suitably, the Bifidobacterium lactis fermentate or Bifidobacterium lactis supernatant (e.g. prior to drying) may comprise about 0.5 wt % or more, about 1 wt % or more, about 1.5 wt % or more, about 2 wt % or more, about 3 wt % or more, about 4 wt % or more, or about 5 wt % or more total acids. Suitably, the Bifidobacterium lactis fermentate or Bifidobacterium lactis supernatant (e.g. prior to drying) may comprise about 0.5 wt % to about 5 wt %, about 1 wt % to about 3 wt %, about 1.5 wt % to about 2.5 wt %, about 2 wt % to about 2.5 wt %, or about 2 wt % total acids.

Suitably, the Bifidobacterium lactis fermentate or Bifidobacterium lactis supernatant (e.g. prior to drying) may comprise about 0.5 wt % to about 6 wt %, about 1 wt % to about 3 wt %, or about 1.5 wt % to about 2.5 wt % ash.

Suitably, the Bifidobacterium lactis fermentate or Bifidobacterium lactis supernatant (e.g. prior to drying) may comprise about 3.5 wt % or less, about 3 wt % or less, about 2.5 wt % or less, about 2 wt % or less, about 1.5 wt % or less, about 1 wt % or less, about 0.8 wt % or less, about 0.6 wt % or less, about 0.4 wt % or less, or about 0.2 wt % or less total amino acids. Suitably, the Bifidobacterium lactis fermentate or Bifidobacterium lactis supernatant (e.g. prior to drying) may comprise about 0.2 wt % to about 3.5 wt %, about 0.4 wt % to about 2 wt %, about 0.6 wt % to about 1 wt %, about 0.7 wt % to about 0.9 wt %, or about 0.8 wt % total amino acids.

Suitably, the Bifidobacterium lactis fermentate or Bifidobacterium lactis supernatant (e.g. prior to drying) may comprise: about 5 wt % or less total sugars; about 0.5 wt % or more total acids; and about 3.5 wt % or less total amino acids.

Suitably, the Bifidobacterium lactis fermentate or Bifidobacterium lactis supernatant (e.g. prior to drying) may comprise: about 2 wt % or less total sugars; about 1 wt % or more total acids; and about 2 wt % or less total amino acids.

Suitably, the Bifidobacterium lactis fermentate or Bifidobacterium lactis supernatant (e.g. prior to drying) may comprise: about 1 wt % or less total sugars; about 1.5 wt % or more total acids; and about 1 wt % or less total amino acids.

Suitably, the Bifidobacterium lactis fermentate or Bifidobacterium lactis supernatant (e.g. prior to drying) may comprise: about 0.5 wt % or less total sugars; about 2 wt % or more total acids; and about 0.8 wt % or less total amino acids.

Suitably, the Bifidobacterium lactis fermentate or Bifidobacterium lactis supernatant (e.g. prior to drying) may comprise: about 0 wt % to about 5 wt % total sugars; about 0.5 wt % to about 5 wt % total acids; and about 0.2 wt % to about 3.5 wt % or less total amino acids.

Suitably, the Bifidobacterium lactis fermentate or Bifidobacterium lactis supernatant (e.g. prior to drying) may comprise: about 0 wt % to about 2 wt % total sugars; about 1 wt % to about 3 wt % total acids; and about 0.4 wt % to about 2 wt % total amino acids.

Suitably, the Bifidobacterium lactis fermentate or Bifidobacterium lactis supernatant (e.g. prior to drying) may comprise: about 0 wt % to about 1 wt % total sugars; about 1.5 wt % to about 2.5 wt % total acids; and about 0.6 wt % to about 1 wt % total amino acids.

Suitably, the Bifidobacterium lactis fermentate or Bifidobacterium lactis supernatant (e.g. prior to drying) may comprise: about 0 wt % to about 0.3 wt % total sugars; about 2 wt % total acids; and about 0.8 wt % total amino acids.

Suitably, the Bifidobacterium lactis fermentate or Bifidobacterium lactis supernatant (e.g. prior to drying) may comprise:
(i) less than about 0.3 wt % glucose;
(ii) less than about 0.3 wt % fructose;
(iii) about 0 wt % to about 0.5 wt % citric acid;
(iv) about 0.6 wt % to about 0.9 wt % lactic acid;
(v) about 0.1 wt % to about 0.3 wt % formic acid;
(vi) about 1.2 wt % to about 1.4 wt % acetic acid; and/or
(vii) about 0.2 wt % total nitrogen.

Suitably, the Bifidobacterium lactis fermentate or Bifidobacterium lactis supernatant (e.g. prior to drying) may comprise: about 0 wt % to about 2 wt % total sugars, including less than about 0.3 wt % glucose and less than about 0.3 wt % fructose; about 1.5 wt % to about 2.5 wt % total acids, including about 0 wt % to about 0.5 wt % citric acid, about 0.6 wt % to about 0.9 wt % lactic acid, about 0.1 wt % to about 0.3 wt % formic acid, and about 1.2 wt % to about 1.4 wt % acetic acid; and about 0.6 wt % to about 1 wt % total amino acids, including about 0.2 wt % total nitrogen.

### Probiotic, Prebiotic, and Synbiotic Combinations

The Bifidobacterium lactis supernatant may be used in combination with one or more probiotics, prebiotics, or synbiotics. In some embodiments, the Bifidobacterium lactis supernatant is used in combination with one or more probiotics. The probiotics, prebiotics, or synbiotics and Bifidobacterium lactis supernatant may be combined in any suitable doses.

In one aspect, the present invention provides a supplement or nutritional composition comprising a combination of a Bifidobacterium lactis supernatant and one or more probiotics, prebiotics, or synbiotics.

In some embodiments, the present invention provides a supplement or nutritional composition comprising a combination of a Bifidobacterium lactis supernatant and one or more probiotics.

The term “probiotic” may refer to a component that contains live microorganisms that, when administered in adequate amounts, confer a health benefit on the subject (see e.g. Hill, C., et al., 2014. Nature reviews Gastroenterology & hepatology, 11 (8), p. 506).

Suitably, the probiotic may comprise a commercially available probiotic strain and/or a strain which has been shown to have health benefits (See e.g. Fijan, S., 2014. International journal of environmental research and public health, 11 (5), pp. 4745-4767). In some embodiments, the probiotic comprises Escherichia, Bifidobacterium, Streptococcus, Lactobacillus (as defined up to March 2020), Bacillus, and/or Enterococcus.

In some embodiments, the probiotic is a Bifidobacterium lactis probiotic. Any suitable Bifidobacterium lactis strain may be used, for example any Bifidobacterium lactis strain which is known to have a probiotic effect. Such Bifidobacterium lactis strains will be well known to the skilled person and are described above in the section entitled “Bifidobacterium lactis strains”. The Bifidobacterium lactis may be the same Bifidobacterium lactis from which the supernatant is derived or may be different. In some embodiments, the Bifidobacterium lactis is the same Bifidobacterium lactis from which the supernatant is derived.

The term “prebiotic” may refer to a non-digestible component that benefits the subject by selectively stimulating the favourable growth and/or activity of one or more microbial taxa. Exemplary prebiotics include human milk oligosaccharides. Exemplary prebiotic oligosaccharides include galacto-oligosaccharides (GOS), fructo-oligosaccharides (FOS), 2′-fucosyllactose, lacto-N-neo-tetraose, and inulin.

The term “synbiotic” may refer to a component that contains both probiotics and prebiotics (see e.g. Swanson, K. S., et al., 2020. Nature Reviews Gastroenterology & Hepatology, 17 (11), pp. 687-701).

### Use as a Bifidogenic Factor

As described above, Bifidobacterium lactis supernatant has surprisingly been found to enhance growth of bifidobacteria, indicating a surprising bifidogenic effect. The supernatant of Bifidobacterium lactis was surprisingly found to enhance growth of bifidobacteria to a significantly greater extent than supernatant of Lactobacillus rhamnosus.

In one aspect, the present invention provides use of a Bifidobacterium lactis supernatant as a bifidogenic factor. As used herein, a “bifidogenic factor” (also known as a “bifidus factor”) is an agent or composition that specifically enhances the growth of bifidobacteria in either a product or in the gastrointestinal tract of humans and/or animals. In some embodiments, the use of a Bifidobacterium lactis supernatant as a bifidogenic factor enhances the growth of bifidobacteria in the gastrointestinal tract of a subject.

In another aspect, the present invention provides a Bifidobacterium lactis supernatant for use in enhancing the growth of bifidobacteria in the gastrointestinal tract of a subject.

The Bifidobacterium lactis supernatant may enhance the growth of one or more bifidobacteria which are usually abundant in the gastrointestinal tract of humans and/or animals. In some embodiments, the Bifidobacterium lactis supernatant enhances the growth of one or more bifidobacteria which are usually abundant in the gastrointestinal tract of humans (see e.g. Turroni, F., et al., 2009. The ISME journal, 3 (6), pp. 745-751 and Turroni, F., et al., 2012. PloS one, 7 (5), p. e36957).

For example, the Bifidobacterium lactis supernatant may enhance the growth of one or more bifidobacterial phylogenetic groups selected from: the B. adolescentis group, the B. bifidum group, the B. longum group, and the B. pseudolongum group.

For example, the Bifidobacterium lactis supernatant may enhance the growth of one or more bifidobacterial species selected from: B. longum, B. animalis, B. adolescentis, B. bifidum, B. catenulatum, B. pseudocatenulatum, B. breve, B. pseudolongum, B. gallicum, B. angulatum,and B. faecal (see e.g. Rivière, A., et al., 2016. Frontiers in microbiology, 7, p. 979). Suitably, the Bifidobacterium lactis supernatant may enhance the growth of B. longum, B. breve, and B. bifidum (typically the most abundant species in infants, see e.g. Arboleya, S., et al., 2016. Frontiers in microbiology, 7, p. 1204) and/or B. catenulatum, B. adolescentis and, B. longum (typically the most abundant species in adults, see e.g. Arboleya, S., et al., 2016. Frontiers in microbiology, 7, p. 1204). Suitably, the Bifidobacterium lactis supernatant may enhance the growth of B. longum.

### Routes of Administration

The Bifidobacterium lactis supernatant, supplement or nutritional composition may be administered by any suitable method known to the skilled person. For example, the Bifidobacterium lactis supernatant, supplement or nutritional composition may be administered by oral and/or enteral administration. In some embodiments, the Bifidobacterium lactis supernatant, supplement or nutritional composition is orally administered.

### Subject

The Bifidobacterium lactis supernatant may be administered to a subject, wherein the subject is a mammal. Suitably, the subject is a human or a pet, such as a dog, a cat, a rodent (e.g. a mouse, a rat, or a guinea pig), or a rabbit. Preferably, the subject is a human subject.

The subject may be any age. For example, the subject may be a child or an adult. The term “child” may refer to a subject aged under 18 years. The term “adult” may refer to a subject aged 18 years or older. In some embodiments, the subject is a child. In some embodiments, the subject is an adult.

In some embodiments, the subject is an infant, a toddler, or a young child. The term “infant” may refer to a subject aged from about 0 years to about 1 year. The term “toddler” may refer to a subject aged from about 1 year to about 3 years. The term “young child” may refer to a subject aged from about 3 years to about 5 years. In some embodiments, the infant, toddler, or young child is a preterm infant, toddler, or young child. A “preterm” or “premature”, toddler, or young child means an infant, toddler, or young child who was not born at term (e.g. born prior 36 weeks of gestation). In some embodiments, the infant, toddler, or young child was born by C-section or was vaginally delivered.

The subject may have or may be at risk of a low abundance of bifidobacteria in their gastrointestinal tract. The abundance of bifidobacteria in the gastrointestinal may be determined by any method known to the skilled person (e.g. any method described in Tang, Q., et al., 2020. Frontiers in cellular and infection microbiology, 10, p. 151).

A gastrointestinal tract sample may be obtained from or obtainable from fecal samples, endoscopy samples (e.g. biopsy samples, luminal brush samples, laser capture microdissection samples), aspirated intestinal fluid samples, surgery samples, or by in vivo models or intelligent capsule. Suitably, a gastrointestinal tract sample may be obtained from or obtainable from fecal samples. Fecal samples are naturally collected, non-invasive and can be sampled repeatedly.

The abundance of bifidobacteria may be determined from the samples by any suitable method. For example, the abundance of bifidobacteria may be obtained by or obtainable from the samples by sequencing methods (e.g. next-generation sequencing (NGS) methods), PCR-based methods, semi-quantitative detection methods, cycling temperature capillary electrophoresis, immunological-based methods, cell-based methods, or any combination thereof.

The subject may have or may be at risk of a disorder associated with decreased numbers of bifidobacteria in the gut. Such disorders are described by Rivière, A., et al., 2016. Frontiers in microbiology, 7, p. 979, and may include gastrointestinal diseases, obesity, allergies, and regressive autism.

The subject may have or may be at risk of a gastrointestinal disease. Such gastrointestinal diseases are described in more detail in the section entitled “Methods of treating and/or preventing a gastrointestinal disease”. In some embodiments, the subject may have or may be at risk of an antibiotic-associated diarrhea. In some embodiments, the subject may have or may be at risk of a Helicobacter pylori infection. In some embodiments, the subject may have or may be at risk of an IBD. In some embodiments, the subject may have or may be at risk of IBS. In some embodiments, the subject may have or may be at risk of lactose intolerance, infectious diarrhea, colorectal cancer, chemotherapy-induced diarrhea, or necrotizing enterocolitis.

In some embodiments, the subject may have or may be at risk of obesity. In some embodiments, the subject may have or may be at risk of allergies. In some embodiments, the subject may have or may be at risk of regressive autism.

### Methods of Treating or Pre Venting Disease

Bifidobacterium is one of the main genera of commensal bacteria present in the human gastrointestinal tracts and its presence has been related to health benefits in several studies (Hidalgo-Cantabrana, C., et al., 2017. Microbiology spectrum, 5 (3), pp. 5-3).

In one aspect, the present invention provides a Bifidobacterium lactis supernatant for use as a medicament. In another aspect, the present invention provides use of a Bifidobacterium lactis supernatant for the manufacture of a medicament. In another aspect, the present invention provides a method of treatment comprising administering a Bifidobacterium lactis supernatant.

In one aspect, the present invention provides a supplement or nutritional composition comprising a Bifidobacterium lactis supernatant for use as a medicament. In another aspect, the present invention provides use of a supplement or nutritional composition comprising a Bifidobacterium lactis supernatant for the manufacture of a medicament. In another aspect, the present invention provides a method of treatment comprising administering a supplement or nutritional composition comprising a Bifidobacterium lactis supernatant.

The Bifidobacterium lactis supernatant, supplement, or nutritional composition may prevent or treat a disease by enhancing the growth of bifidobacteria in the gastrointestinal tract of the subject.

The Bifidobacterium lactis supernatant may be used to treat or prevent disorders associated with decreased numbers of bifidobacteria in the gut (see e.g. Rivière, A., et al., 2016. Frontiers in microbiology, 7, p. 979).

In one aspect, the invention provides a Bifidobacterium lactis supernatant for use in treating and/or preventing a disorder associated with decreased numbers of bifidobacteria in the gut. In another aspect, the invention provides for use of a Bifidobacterium lactis supernatant for the manufacture of a medicament for treating and/or preventing a disorder associated with decreased numbers of bifidobacteria in the gut. In another aspect, the invention provides a method of treating and/or preventing a disorder associated with decreased numbers of bifidobacteria in the gut in a subject, comprising administering a Bifidobacterium lactis supernatant to the subject.

In one aspect, the invention provides a supplement or nutritional composition comprising a Bifidobacterium lactis supernatant for use in treating and/or preventing a disorder associated with decreased numbers of bifidobacteria in the gut. In another aspect, the invention provides a supplement or nutritional composition comprising a Bifidobacterium lactis supernatant for the manufacture of a medicament for treating and/or preventing a disorder associated with decreased numbers of bifidobacteria in the gut. In another aspect, the invention provides a method of treating and/or preventing a disorder associated with decreased numbers of bifidobacteria in the gut in a subject, comprising administering a supplement or nutritional composition comprising a Bifidobacterium lactis supernatant to the subject.

Suitably, the disorder associated with decreased numbers of bifidobacteria in the gut in a subject may be selected from: a gastrointestinal disease, obesity, an allergic disease, and regressive autism.

### Methods of Promoting and/or Maintaining Gastrointestinal Health

As described above, the use of probiotics, including bifidobacteria strains, in preventive medicine to maintain a healthy intestinal function is well-documented (Tojo, R., et al., 2014. World journal of gastroenterology: WJG, 20 (41), p. 15163). Postbiotics which enhance the growth of probiotic strains such as bifidobacteria can be expected to have similar effects on gastrointestinal health.

In one aspect, the invention provides a Bifidobacterium lactis supernatant for use in promoting and/or maintaining gastrointestinal health. In another aspect, the invention provides for use of a Bifidobacterium lactis supernatant for the manufacture of a medicament for promoting and/or maintaining gastrointestinal health. In another aspect, the invention provides a method of promoting and/or maintaining gastrointestinal health of a subject, comprising administering a Bifidobacterium lactis supernatant to the subject.

In one aspect, the invention provides a supplement or nutritional composition comprising a Bifidobacterium lactis supernatant for use in promoting and/or maintaining gastrointestinal health. In another aspect, the invention provides a supplement or nutritional composition comprising a Bifidobacterium lactis supernatant for the manufacture of a medicament for promoting and/or maintaining gastrointestinal health. In another aspect, the invention provides a method of promoting and/or maintaining gastrointestinal health of a subject, comprising administering a supplement or nutritional composition comprising a Bifidobacterium lactis supernatant to the subject.

In one aspect, the invention provides use of a Bifidobacterium lactis supernatant in promoting and/or maintaining gastrointestinal health. In another aspect, the invention provides use of a supplement or nutritional composition comprising a Bifidobacterium lactis supernatant in promoting and/or maintaining gastrointestinal health.

The Bifidobacterium lactis supernatant, supplement, or nutritional composition may promote and/or maintain gastrointestinal health by enhancing the growth of bifidobacteria in the gastrointestinal tract of the subject.

### Methods of Treating and/or Preventing a Gastrointestinal Disease

Beneficial effects resulting from the consumption of bifidobacteria on human health have been associated with the prevention and treatment of gastrointestinal diseases (Hidalgo-Cantabrana, C., et al., 2017. Microbiology spectrum, 5 (3), pp. 5-3). Postbiotics which enhance the growth of bifidobacteria can be expected to have similar effects.

In one aspect, the invention provides a Bifidobacterium lactis supernatant for use in treating and/or preventing a gastrointestinal disease. In another aspect, the invention provides for use of a Bifidobacterium lactis supernatant for the manufacture of a medicament for treating and/or preventing a gastrointestinal disease. In another aspect, the invention provides a method of treating and/or preventing a gastrointestinal disease in a subject, comprising administering a Bifidobacterium lactis supernatant to the subject.

In one aspect, the invention provides a supplement or nutritional composition comprising a Bifidobacterium lactis supernatant for use in treating and/or preventing a gastrointestinal disease. In another aspect, the invention provides a supplement or nutritional composition comprising a Bifidobacterium lactis supernatant for the manufacture of a medicament for treating and/or preventing a gastrointestinal disease. In another aspect, the invention provides a method of treating and/or preventing a gastrointestinal disease in a subject, comprising administering a supplement or nutritional composition comprising a Bifidobacterium lactis supernatant to the subject.

As used herein, a “gastrointestinal disease” (also known as “GI disease” or “GI illness”) may refer to diseases involving the gastrointestinal tract, which includes the oesophagus, stomach, small intestine, large intestine and rectum. In some embodiments, the gastrointestinal disease is a gastric disease or an intestinal disease.

In some embodiments, the gastrointestinal disease is a gastric disease. A “gastric disease” may refer to diseases affecting the stomach.

In some embodiments, the gastrointestinal disease is an intestinal disease. An “intestinal disease” may refer to diseases affecting the small intestine (including the duodenum, jejunum, and ileum) or large intestine (including the cecum, colon, and rectum).

In some embodiments, the gastrointestinal disease is selected from: antibiotic-associated diarrhea, Helicobacter pylori infection, an inflammatory bowel disease (IBD), irritable bowel syndrome (IBS), lactose intolerance, infectious diarrhea, colorectal cancer, chemotherapy-induced diarrhea, and necrotizing enterocolitis.

In some embodiments, the gastrointestinal disease is selected from: antibiotic-associated diarrhea, Helicobacter pylori infection, an inflammatory bowel disease (IBD), and irritable bowel syndrome (IBS).

### Antibiotic-Associated Diarrhea

A common complication of antibiotic use is the development of gastrointestinal disease. This complication ranges from mild diarrhea to pseudomembranous colitis. Antibiotic-associated diarrhea typically occurs in 5-35% of patients taking antibiotics and varies depending upon the specific type of antibiotic, the health of the host and exposure to pathogens. The pathogenesis of antibiotic-associated diarrhea may be mediated through the disruption of the normal microbiota resulting in pathogen overgrowth or metabolic imbalances (McFarland, L.V., 2008. Future Microbiology, 3 (5), p. 563).

A probiotic mixture which contains several bifidobacteria strains, among which are B. breve, B. infantis, and B. longum, displayed an ability to reduce the incidence of antibiotic-associated diarrhea (Selinger, C. P., et al., 2013. Journal of Hospital Infection, 84 (2), pp. 159-165).

In one aspect, the invention provides a Bifidobacterium lactis supernatant for use in treating and/or preventing an antibiotic-associated diarrhea. In another aspect, the invention provides for use of a Bifidobacterium lactis supernatant for the manufacture of a medicament for treating and/or preventing an antibiotic-associated diarrhea. In another aspect, the invention provides a method of treating and/or preventing an antibiotic-associated diarrhea in a subject, comprising administering a Bifidobacterium lactis supernatant to the subject.

In one aspect, the invention provides a supplement or nutritional composition comprising a Bifidobacterium lactis supernatant for use in treating and/or preventing an antibiotic-associated diarrhea. In another aspect, the invention provides a supplement or nutritional composition comprising a Bifidobacterium lactis supernatant for the manufacture of a medicament for treating and/or preventing an antibiotic-associated diarrhea. In another aspect, the invention provides a method of treating and/or preventing an antibiotic-associated diarrhea in a subject, comprising administering a supplement or nutritional composition comprising a Bifidobacterium lactis supernatant to the subject.

### Helicobacter pylori Infection

Helicobacter pylori is a gram-negative microaerophilic bacterium that infects the epithelial lining of the stomach. Helicobacter pylori is the main cause of chronic gastritis and the principal etiological agent for gastric cancer and peptic ulcer disease. A recent global systematic review estimated that more than half the world's population is infected with Helicobacter pylori (Hooi, J. K., et al., 2017. Gastroenterology, 153 (2), pp. 420-429).

A Helicobacter pylori eradication rate of 32.5% has been reported in adults after 10 days of administration of a probiotic mixture which contains several bifidobacteria strains, among which are B. breve, B. infantis, and B. longum (Boltin, D., 2016. Best Practice & Research Clinical Gastroenterology, 30 (1), pp. 99-109). Moreover, such a probiotic mixture has be shown to accelerate gastric ulcer healing (Dharmani, P., et al., 2013. PLOS One, 8 (3), p. e58671).

In one aspect, the invention provides a Bifidobacterium lactis supernatant for use in treating and/or preventing a Helicobacter pylori infection. In another aspect, the invention provides for use of a Bifidobacterium lactis supernatant for the manufacture of a medicament for treating and/or preventing a Helicobacter pylori infection. In another aspect, the invention provides a method of treating and/or preventing a Helicobacter pylori infection in a subject, comprising administering a Bifidobacterium lactis supernatant to the subject.

In one aspect, the invention provides a supplement or nutritional composition comprising a Bifidobacterium lactis supernatant for use in treating and/or preventing a Helicobacter pylori infection. In another aspect, the invention provides a supplement or nutritional composition comprising a Bifidobacterium lactis supernatant for the manufacture of a medicament for treating and/or preventing a Helicobacter pylori infection. In another aspect, the invention provides a method of treating and/or preventing a Helicobacter pylori infection in a subject, comprising administering a supplement or nutritional composition comprising a Bifidobacterium lactis supernatant to the subject.

### Inflammatory Bowel Disease

Inflammatory bowel disease (IBD) is a group of inflammatory conditions of the colon and small intestine. Exemplary IBDs include Crohn's disease (CD), ulcerative colitis (UC), and pouchitis. Is has been suggested that dysbiosis (that is, abnormal microbiota composition) and decreased complexity of the gut microbial ecosystem are common features in patients with IBD (see Manichanh, C., et al., 2012. Nature reviews Gastroenterology & hepatology, 9 (10), pp. 599-608).

In IBD, a probiotic mixture which contains several bifidobacteria strains, among which are B. breve, B. infantis, and B. longum, was able to reduce the UC symptoms in adults (Tursi, A., et al., 2010. The American journal of gastroenterology, 105 (10), p. 2218) as well as the remission of the disease in children (Miele, E., et al., 2009. American Journal of Gastroenterology, 104 (2), pp. 437-443).

In one aspect, the invention provides a Bifidobacterium lactis supernatant for use in treating and/or preventing an IBD. In another aspect, the invention provides for use of a Bifidobacterium lactis supernatant for the manufacture of a medicament for treating and/or preventing an IBD. In another aspect, the invention provides a method of treating and/or preventing an IBD in a subject, comprising administering a Bifidobacterium lactis supernatant to the subject.

In one aspect, the invention provides a supplement or nutritional composition comprising a Bifidobacterium lactis supernatant for use in treating and/or preventing an IBD. In another aspect, the invention provides a supplement or nutritional composition comprising a Bifidobacterium lactis supernatant for the manufacture of a medicament for treating and/or preventing an IBD. In another aspect, the invention provides a method of treating and/or preventing an IBD in a subject, comprising administering a supplement or nutritional composition comprising a Bifidobacterium lactis supernatant to the subject.

In some embodiments, the IBD is Crohn's disease, ulcerative colitis, or pouchitis.

### Irritable Bowel Syndrome

Irritable bowel syndrome (IBS) is a functional bowel disorder characterised by chronic and recurrent abdominal pain and altered bowel habit (Chey, W. D., et al., 2015. Jama, 313 (9), pp. 949-958). A growing body of evidence indicates dysbiosis as a hallmark of IBS (Rodiño-Janeiro, B. K., et al., 2018. Advances in therapy, 35 (3), pp. 289-310).

Administration of a probiotic mixture which contains several bifidobacteria strains, among which are B. breve, B. infantis, and B. longum, for 6 weeks resulted in the reduction of IBS symptoms and the improvement of the quality of life in children (Guandalini, S., et al., 2010. Journal of pediatric gastroenterology and nutrition, 51 (1), pp. 24-30).

In one aspect, the invention provides a Bifidobacterium lactis supernatant for use in treating and/or preventing IBS. In another aspect, the invention provides for use of a Bifidobacterium lactis supernatant for the manufacture of a medicament for treating and/or preventing IBS. In another aspect, the invention provides a method of treating and/or preventing IBS in a subject, comprising administering a Bifidobacterium lactis supernatant to the subject.

In one aspect, the invention provides a supplement or nutritional composition comprising a Bifidobacterium lactis supernatant for use in treating and/or preventing IBS. In another aspect, the invention provides a supplement or nutritional composition comprising a Bifidobacterium lactis supernatant for the manufacture of a medicament for treating and/or preventing IBS. In another aspect, the invention provides a method of treating and/or preventing IBS in a subject, comprising administering a supplement or nutritional composition comprising a Bifidobacterium lactis supernatant to the subject.

### Other Gastrointestinal Diseases

Lactose intolerance is a common condition caused by a decreased ability to digest lactose. Administration of bifidobacteria has been used to improve the symptoms of lactose intolerance (Hidalgo-Cantabrana, C., et al., 2017. Microbiology spectrum, 5 (3), pp. 5-3).

Infectious diarrhea (also known as gastroenteritis) is inflammation of the gastrointestinal tract caused by an infection. Gastroenteritis is usually caused by viruses (e.g. rotavirus, norovirus, adenovirus, astrovirus, and coronavirus), however, bacteria (e.g. C. jeuni, E. coli, Salmonella, Shigella, C. difficile, and S. aureus), parasites (e.g. Giardia lamblia), and fungus can also cause gastroenteritis. There is evidence that that viable Bifidobacterium lactis has some protective effect against acute diarrhea in healthy children (Chouraqui, J. P., et al., 2004. Journal of pediatric gastroenterology and nutrition, 38 (3), pp. 288-292).

In colorectal cancer patients, it is known that the composition of the microbiota is one of the factors favouring the development of carcinogenic lesions. Probiotics have been used to modulate the microbiota in colorectal cancer (Hidalgo-Cantabrana, C., et al., 2017. Microbiology spectrum, 5 (3), pp. 5-3).

Patients receiving cytotoxic and radiation therapy exhibit marked changes in intestinal microbiota, e.g. a decrease in Bifidobacterium. These modifications may contribute to the development of chemotherapy-induced diarrhoea (Touchefeu, Y., et al., 2014. Alimentary pharmacology & therapeutics, 40 (5), pp. 409-421).

Necrotizing enterocolitis (NEC) is an intestinal disease that affects premature infants. It has been shown that probiotic supplementation with bifidobacteria can reduce both the incidence and severity of NEC in a premature neonatal population (Bin-Nun, A., et al., 2005. The Journal of pediatrics, 147 (2), pp. 192-196).

In one aspect, the invention provides a Bifidobacterium lactis supernatant for use in treating and/or preventing lactose intolerance, infectious diarrhea, colorectal cancer, chemotherapy-induced diarrhea, or necrotizing enterocolitis. In another aspect, the invention provides for use of a Bifidobacterium lactis supernatant for the manufacture of a medicament for treating and/or preventing lactose intolerance, infectious diarrhea, colorectal cancer, chemotherapy-induced diarrhea, or necrotizing enterocolitis. In another aspect, the invention provides a method of treating and/or preventing lactose intolerance, infectious diarrhea, colorectal cancer, chemotherapy-induced diarrhea, or necrotizing enterocolitis in a subject, comprising administering a Bifidobacterium lactis supernatant to the subject.

In one aspect, the invention provides a supplement or nutritional composition comprising a Bifidobacterium lactis supernatant for use in treating and/or preventing lactose intolerance, infectious diarrhea, colorectal cancer, chemotherapy-induced diarrhea, or necrotizing enterocolitis. In another aspect, the invention provides a supplement or nutritional composition comprising a Bifidobacterium lactis supernatant for the manufacture of a medicament for treating and/or preventing lactose intolerance, infectious diarrhea, colorectal cancer, chemotherapy-induced diarrhea, or necrotizing enterocolitis. In another aspect, the invention provides a method of treating and/or preventing lactose intolerance, infectious diarrhea, colorectal cancer, chemotherapy-induced diarrhea, or necrotizing enterocolitis in a subject, comprising administering a supplement or nutritional composition comprising Bifidobacterium lactis supernatant to the subject.

### Methods of Manufacture

The Bifidobacterium lactis supernatant of the present invention may be prepared by any suitable method known in the art. For example, preparation of bacterial supernatants is described in Moradi, M., et al., 2021. Enzyme and Microbial Technology, 143, p. 109722.

Exemplary culture conditions and processing steps are described above in the section entitled “Supernatant preparation”, and the methods of manufacture according to the present invention may include any of the steps described therein.

In one aspect, the present invention provides a method of manufacturing a Bifidobacterium lactis supernatant, the method comprising:
(a) culturing a Bifidobacterium lactis in a culture media to provide a Bifidobacterium lactis fermentate;
(b) removing substantially all the Bifidobacterium lactis cells from the Bifidobacterium lactis fermentate to provide a Bifidobacterium lactis supernatant, preferably wherein the Bifidobacterium lactis cells are removed by centrifugation; and
(c) optionally, pasteurising the Bifidobacterium lactis supernatant.

The method may comprise any other suitable processing steps. In preferred embodiments, the method further comprises a step (d) of drying the Bifidobacterium lactis supernatant.

The Bifidobacterium lactis supernatant may be any Bifidobacterium lactis supernatant described herein.

In one aspect, the present invention provides a method of providing a supplement comprising a Bifidobacterium lactis supernatant, the method comprising:
(a) culturing a Bifidobacterium lactis in a culture media to provide a Bifidobacterium lactis fermentate;
(b) removing substantially all the Bifidobacterium lactis cells from the Bifidobacterium lactis fermentate to provide a Bifidobacterium lactis supernatant, preferably wherein the Bifidobacterium lactis cells are removed by centrifugation;
(c) optionally, pasteurising the Bifidobacterium lactis supernatant;
(d) drying the Bifidobacterium lactis supernatant to provide a Bifidobacterium lactis supernatant powder; and
(e) processing the Bifidobacterium lactis supernatant powder to provide a supplement comprising the Bifidobacterium lactis supernatant powder.

The method may comprise any other suitable processing steps. For example, in some embodiments, step (e) comprises a step of encapsulating, compressing and/or packaging the Bifidobacterium lactis supernatant powder to provide the supplement. For example, in some embodiments step (e) comprises a step of encapsulating the Bifidobacterium lactis supernatant powder to provide a capsule comprising the Bifidobacterium lactis supernatant.

The supplement may be any supplement described herein.

Exemplary culture conditions and processing steps are described above in the section entitled “Supernatant preparation”, and the methods of manufacture according to the present invention may include any of the steps described therein.

### EXAMPLES

The invention will now be further described by way of Examples, which are meant to serve to assist one of ordinary skill in the art in carrying out the invention and are not intended in any way to limit the scope of the invention.

### Example 1—Bifidogenic Effect of B. lactis vs L. rhamnosus Supernatant

### Production of Supernatants

Supernatants of B. lactis (Bifidobacterium animalis subsp. lactis CNCM I-3446) and of L. rhamnosus (Lacticaseibacillus rhamnosus CGMCC 1.3724) were produced at laboratory scale.

Both strains were grown first as an overnight culture in a bottle to produce sufficient inoculum for a fermentation step. The fermentation step was carried out in 1.6 L scale in fermenters at 37° C. with pH control, stirring and head space gassing until stationary phase was reached. For B. lactis anaerobic conditions (carbon dioxide headspace) were employed, pH was controlled at 6.0 and the media contained yeast extract, sodium ascorbate and glucose syrup. For L. rhamnosus air headspace was used, the pH was controlled at 5.5 and the media contained yeast extract/peptone, manganese sulphate, fructose and glucose. Optionally, polysorbate 80 can be added to the media, but it has no impact on the growth of the two strains.

After reaching stationary phase, the fermentates were centrifuged to remove the vast majority of the bacterial cells and the supernatants were either analysed directly (e.g. for cell counts) or frozen for further analysis later. Analytical results are summarized in Table 1.

TABLE 1
Characterization of the supernatants obtained from fermentation
with B. lactis (Bifidobacterium animalis subsp. lactis CNCM
I-3446) and L. rhamnosus (Lacticaseibacillus rhamnosus CGMCC
1.3724) at lab scale. The range for cell counts reflects the
range observed in replicates of independent trials.

| | Supernatant | Supernatant |
|---|---|---|
| | L. rhamnosus | B. lactis |
| Cell count by plating, before | 2E+07-2E+08 | 1E+08-7E+08 |
| heat treatment (cfu/mL) | | |
| pH | 5.5 | 6.2 |
| Total solids (%) | 9.8 | 6.1 |
| Glucose (%) | 0.01 | 0.05 |
| Fructose (%) | 0.01 | 0.01 |
| Citric acid (%) | 0.01 | 0.01 |
| Lactic acid (%) | 5.35 | 0.61 |
| Formic acid (%) | 0.09* | 0.33* |
| Acetic acid (%) | 0.06 | 1.26 |
| Ash (%) | 3.6 | 2.4 |
| Total nitrogen (%) | 0.22 | 0.19 |

*Probably overestimated due to overlapping peaks

The supernatants were heat treated at 80° C. for 10 seconds to inactivate the remaining live cells before testing the supernatants in an in vitro model of the gastrointestinal tract.

### Short-Term Colonic Incubations

To assess a potential impact of the two different supernatants on adult intestinal microbiota, an in vitro model of the colon was employed. Short-term batch experiments represent a simplified simulation of the continuous Simulator of the Human Microbial Ecosystem (SHIME®), a model which has been used for over 20 years and has been validated with in vivo parameters. Short-term colonic incubation assays typically consist of a colonic fermentation of a selected dose of the test compound(s) under simulated conditions representative for the proximal large intestine of a healthy adult, using a bacterial inoculum obtained from selected donors.

Freshly prepared human faecal samples were used as a source of the microbial community for inoculation of the colonic models. Faecal inocula were obtained from three different healthy donors (donors A, B, C). At the start of the short-term colonic incubations, the test products were added to sugar-depleted nutritional medium containing basal nutrients that are present in the colon (e.g. host-derived glycans such as mucin). The dose for B. lactis supernatant was 7.29 mL/70 mL incubation and 4.49 mL/70 mL for L. rhamnosus supernatant. The dose was standardized for same total solids addition to facilitate a direct comparison of the two supernatants. A blank was also included, containing only sugar-depleted nutritional medium to assess the background activity of the community. This procedure allowed to assess the specific effects of test ingredients on the metabolic and community composition profile of the colonic microbiota. Incubations were performed during 48 h, at 37° C., under shaking (90 rpm) and anaerobic conditions. In order to account for biological variability, all tests were performed in triplicate.

### Changes in the Microbial Community Composition (qPCR)

qPCR targets the 16S rRNA gene that consists of variable and conserved regions. The nine variable regions (V1-V9) are characterized by a much higher evolutionary rate than the conserved regions. These variable gene regions are typically used to distinguish between different taxonomic groups of bacteria. In this study selective primers for bifidobacteria were used, allowing the direct targeted quantification of this taxonomic group of interest within the microbial ecosystem. As this technique is not dependent on the (lack of) culturability of bacteria, data generated with this method offer a reliable insight in the quantitative effects of a treatment on the microbial community.

Bifidobacteria are regarded as beneficial saccharolytic bacteria, capable of producing high concentrations of lactate and acetate. Lactate is an important metabolite because of its antimicrobial properties, but also (together with acetate) because it is the driver of a series of trophic interactions with other bacteria, resulting in the production of downstream metabolites.

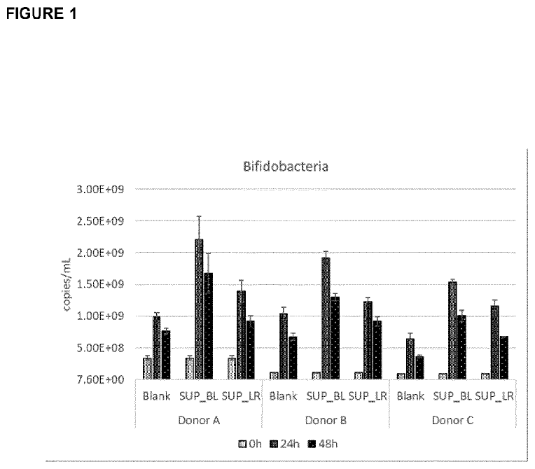

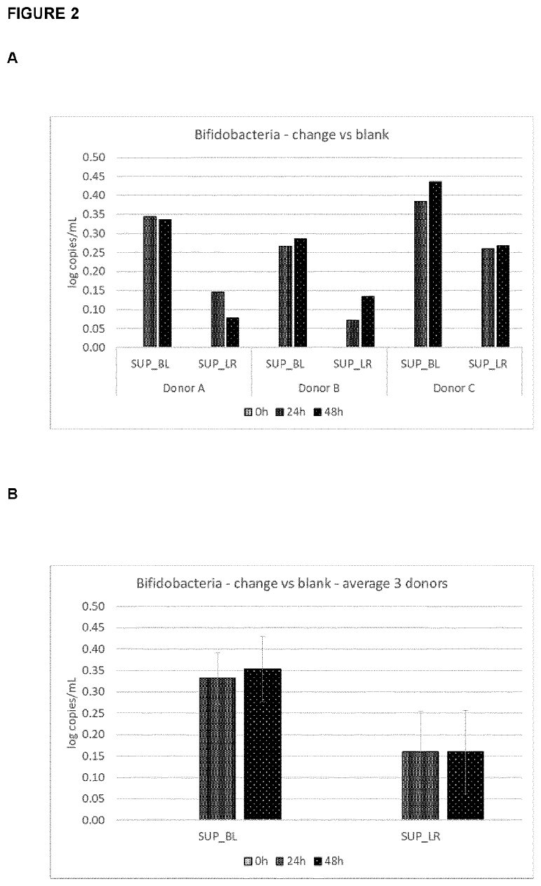

In both supernatants, sugars had essentially been depleted by B. lactis or L. rhamnosus during fermentation (see also sugar concentrations in supernatants, Table 1) and both strains had stopped growing and producing acids due to a lack of a usable carbon source (stationary phase). When feeding these sugar depleted supernatants to the colon model it was thus very surprising to find an increase of bifidobacteria that was even more pronounced for the B. lactis supernatant than for the L. rhamnosus supernatant (FIGS. 1 and 2).

FIG. 1 shows that bifidobacteria increase for all variants and all donors after 24 h of incubation and then slightly decrease at 48 h. The increase of bifidobacteria indicates growth during the first 24 h of incubation. The increase is much more pronounced for the supernatants than for the blank. The difference of increase in bifidobacteria versus the blank incubations is shown in FIG. 2. B. lactis supernatant is clearly superior to L. rhamnosus supernatant in inducing an increase of bifidobacteria for all 3 donors. The difference versus the blank remains at the same level at 48 h.

The increase of bifidobacteria indicates growth and thus a surprising bifidogenic effect of the supernatants. This bifidogenic effect of B. lactis supernatant was superior to L. rhamnosus supernatant for all donors.

### Example 2—Bifidogenic Effect of B. lactis Supernatant Versus B. lactis Probiotic Versus a Combination of Both

### Production of Supernatant Powder

Supernatant of B. lactis (Bifidobacterium animalis subsp. lactis CNCM I-3446) was produced at pilot plant scale (8000 L main fermentation). The inoculum for the main fermentation was produced at pilot plant scale in a 1200 L starter fermentation. The main fermentation step was carried out at 37° C. with pH control (pH 6.0), stirring and head space gassing (carbon dioxide) until stationary phase was reached. The media contained yeast extract, sodium ascorbate and dextrose. After reaching stationary phase, the fermentate was centrifuged with a continuous centrifuge to remove the vast majority of the bacterial cells. Subsequently, the supernatant was pasteurized with a plate heat exchanger at 99° C. for 10 seconds to inactivate the remaining live cells before filling into canisters and freezing.

To convert the liquid supernatant into a powder that could be used for example in supplements, a drying process was developed. Spray drying was selected as preferred technology as this technology is suitable for large volumes, it is energy efficient and readily available. For spray drying experiments a table top Büchi spray dryer was used. Carrier materials were tested that would reduce stickiness and improve physical stability. Suitable food grade carrier materials identified in these tests were oat fibre, maltodextrin, acacia gum (gum arabic), starch and inulin. The carriers were added to the supernatant solutions with total solids ratios of 1:3 and 1:1 (carrier: supernatant dry solids). Overall the ratio of 1:1 resulted in less sticky behaviour compared to the 1:3 ratio.

Out of all the powders produced the supernatant powder with acacia gum with 1:1 ratio was selected for an in vitro colon simulation study.

### Short-Term Colonic Incubations

To assess a potential impact of the supernatant powder with or without the probiotic B. lactis (Bifidobacterium animalis subsp. lactis CNCM I-3446) on adult intestinal microbiota, an in vitro model of the colon as in Example 1 was employed.

Freshly prepared human faecal samples were used as a source of the microbial community for inoculation of the colonic models. Faecal inocula were obtained from three different healthy donors (donors A, B, C; not the same as in Example 1). At the start of the short-term colonic incubations, the test products were added to sugar-depleted nutritional medium containing basal nutrients that are present in the colon (e.g. host-derived glycans such as mucin). The dose for B. lactis supernatant powder was 3.6 g/L and for B. lactis probiotic culture powder 1.4E+08 cfu/mL. The two ingredients were used individually and in combination. A blank was also included, containing only sugar-depleted nutritional medium to assess the background activity of the community. This procedure allowed to assess the specific effects of test ingredients on the metabolic and community composition profile of the colonic microbiota. Incubations were performed during 48 h, at 37° C., under shaking (90 rpm) and anaerobic conditions. In order to account for biological variability, all tests were performed in triplicate.

### Changes in the Microbial Community Composition (qPCR)

As in Example 1, selective qPCR for bifidobacteria was used to analyse changes induced by the treatments in the faecal microbial community of the three donors.

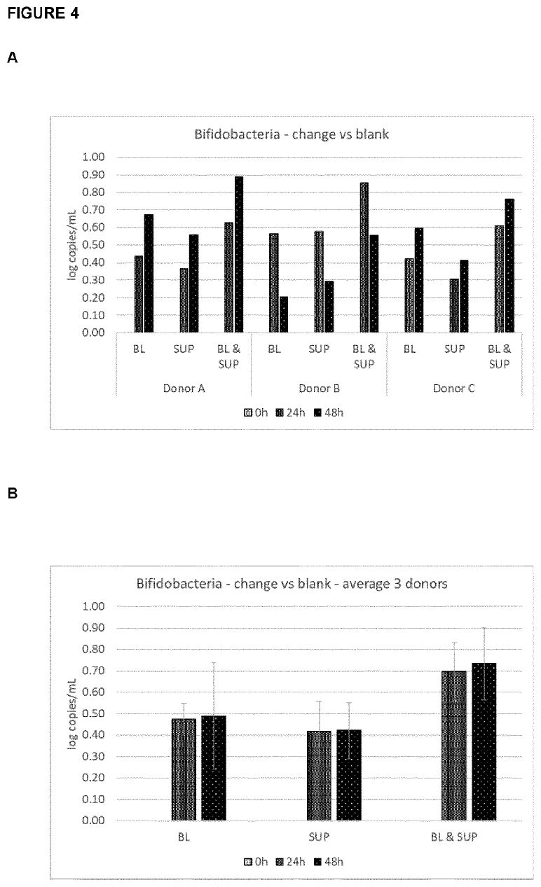

Figurer 3 shows that bifidobacteria increase for all variants and all donors after 24 h of incubation and then slightly decrease at 48 h. The increase is much more pronounced for the treatments than for the blank. The difference of increase in bifidobacteria versus the blank incubations is shown in FIG. 4. The increase in bifidobacteria obtained with the supernatant powder was comparable to that of the 1.4E+08 cfu/mL B. lactis probiotic addition, i.e. the supernatant powder had a bifidogenic effect comparable to adding a live probiotic. The bifidogenic effect of the pure liquid supernatant of B. lactis (Example 1) was thus maintained when turning the supernatant into a powder. The strongest increase in bifidobacteria was achieved with a combination of supernatant powder and B. lactis probiotic, indicating a synergistic effect.

### EMBODIMENTS

Various preferred features and embodiments of the present invention will now be described with reference to the following numbered paragraphs (paras).
1. Use of a Bifidobacterium lactis supernatant as a bifidogenic factor.
2. A Bifidobacterium lactis supernatant for use in enhancing the growth of bifidobacteria in the gastrointestinal tract of a subject.
3. A Bifidobacterium lactis supernatant for use in treating or preventing a gastrointestinal disease by enhancing the growth of bifidobacteria in the gastrointestinal tract of a subject.
4. The use according to para 1, or the Bifidobacterium lactis supernatant for use according to para 2 or 3, wherein the Bifidobacterium lactis is selected from: Bifidobacterium animalis subsp. lactis CNCM I-3446, Bifidobacterium animalis subsp. lactis BI12, Bifidobacterium animalis subsp. lactis BLC1, Bifidobacterium animalis subsp. lactis DSM10140, Bifidobacterium animalis subsp. lactis V9, Bifidobacterium animalis subsp. lactis BI-04, Bifidobacterium animalis subsp. lactis Bi-07, Bifidobacterium animalis subsp. lactis B420, Bifidobacterium animalis subsp. lactis BB-12, Bifidobacterium animalis subsp. lactis AD011, Bifidobacterium animalis subsp. lactis HN019, Bifidobacterium animalis subsp. lactis DN-173 010, Bifidobacterium animalis subsp. lactis ATCC 27536, and Bifidobacterium animalis subsp. lactis VTT E-012010.
5. The use according to para 1 or 4, or the Bifidobacterium lactis supernatant for use according to any of paras 2 to 4, wherein the Bifidobacterium lactis is a Bifidobacterium animalis subsp. lactis having at least 99.0%, at least 99.1%, at least 99.2%, at least 99.3%, at least 99.4%, at least 99.5%, at least 99.6%, at least 99.7%, at least 99.8%, or at least 99.9% sequence identity to Bifidobacterium animalis subsp. lactis CNCM I-3446.
6. The use according to any of paras 1, 4, or 5, or the Bifidobacterium lactis supernatant for use according to any of paras 2 to 5, wherein the Bifidobacterium lactis is Bifidobacterium animalis subsp. lactis CNCM I-3446.
7. The use according to any of paras 1 or 4 to 6, or the Bifidobacterium lactis supernatant for use according to any of paras 2 to 6, wherein the Bifidobacterium lactis supernatant is obtained or obtainable by culturing Bifidobacterium lactis in a culture media comprising sugar and yeast extract, and, optionally, sodium ascorbate and/or polysorbate.
8. The use according to para 7, or the Bifidobacterium lactis supernatant for use according to para 7, wherein:
   (i) the culture media comprises about 1 wt % to about 6 wt %, or about 2 wt % to about 4 wt % sugar;
   (ii) the culture media comprises about 1 wt % to about 10 wt %, or about 1 wt % to about 6 wt %, or about 2 wt % to about 4 wt % yeast extract;
   (iii) the culture media comprises about 0 wt % to about 0.5 wt %, or about 0.1 wt % to about 0.2 wt % sodium ascorbate; and
   (iv) the culture media comprises about 0 wt % to about 1 wt %, or about 0 wt % to about 0.3 wt % polysorbate.
9. The use according to para 7 or 8, or the Bifidobacterium lactis supernatant for use according to para 7 or 8, wherein the sugar is glucose, dextrose and/or glucose syrup.
10. The use according to any of paras 7 to 9, or the Bifidobacterium lactis supernatant for use according to any of paras 7 to 9, wherein the Bifidobacterium lactis supernatant is obtained or obtainable by culturing at a pH of from about 5 to about 7, a pH of from about 5.5 to about 6.5, or a pH of about 6.
11. The use according to any of paras 1 or 4 to 10, or the Bifidobacterium lactis supernatant for use according to any of paras 2 to 10, wherein the Bifidobacterium lactis supernatant is obtained or obtainable by culturing the Bifidobacterium lactis until stationary phase is reached.
12. The use according to any of paras 1 or 4 to 11, or the Bifidobacterium lactis supernatant for use according to any of paras 2 to 11, wherein the Bifidobacterium lactis supernatant is obtained or obtainable by culturing the Bifidobacterium lactis under anaerobic conditions.
13. The use according to any of paras 1 or 4 to 12, or the Bifidobacterium lactis supernatant for use according to any of paras 2 to 12, wherein the Bifidobacterium lactis supernatant is obtained or obtainable by removing substantially all the Bifidobacterium lactis cells from a Bifidobacterium lactis fermentate.
14. The use according to any of paras 1 or 4 to 13, or the Bifidobacterium lactis supernatant for use according to any of paras 2 to 13, wherein the Bifidobacterium lactis supernatant is pasteurised.
15. The use according to any of paras 1 or 4 to 14, or the Bifidobacterium lactis supernatant for use according to any of paras 2 to 14, wherein the Bifidobacterium lactis supernatant is dried.
16. The use according to para 15, or the Bifidobacterium lactis supernatant for use according to para 15, wherein the Bifidobacterium lactis supernatant is dried by spray-drying.
17. The use according to para 16, or the Bifidobacterium lactis supernatant for use according to para 16, wherein the Bifidobacterium lactis supernatant is spray-dried with a carrier material selected from one or more of: oat fibre, maltodextrin, acacia gum, starch and inulin, preferably wherein the Bifidobacterium lactis supernatant is spray-dried with acacia gum
18. The use according to para 17, or the Bifidobacterium lactis supernatant for use according to para 17, wherein the Bifidobacterium lactis supernatant and carrier material are mixed in a total solids ratio of from about 1:3 to about 2:1 (carrier: supernatant dry solids), preferably wherein the total solids ratio is about 1:1 (carrier: supernatant dry solids).
19. The use according to any of paras 1 or 4 to 18, or the Bifidobacterium lactis supernatant for use according to any of paras 2 to 18, wherein the Bifidobacterium lactis supernatant is administered orally.
20. The use according to any of paras 1 or 4 to 19, or the Bifidobacterium lactis supernatant for use according to any of paras 2 to 19, wherein the Bifidobacterium lactis supernatant is in the form of a supplement or a nutritional composition.
21. The use according to any of paras 1 or 4 to 20, or the Bifidobacterium lactis supernatant for use according to any of paras 2 to 20, wherein the Bifidobacterium lactis supernatant is in the form of a capsule or a tablet.
22. The use according to any of paras 1 or 4 to 21, or the Bifidobacterium lactis supernatant for use according to any of paras 2 to 21, wherein compared to Bifidobacterium lactis culture media the Bifidobacterium lactis supernatant has:
   (i) a decreased concentration of total sugar;
   (ii) an increased concentration of total acids; and/or
   (iii) a decreased concentration of total amino acids.
23. The use according to para 22, or the Bifidobacterium lactis supernatant for use according to para 22, wherein compared to Bifidobacterium lactis culture media the concentration of total sugar has decreased by at least about 50%, at least about 60%, at least about 70%, at least about 80%, or at least about 90%.
24. The use according to para 22 or 23, or the Bifidobacterium lactis supernatant for use according to para 22 or 23, wherein compared to Bifidobacterium lactis culture media the concentration of total acids has increased by at least about 70%, by at least about 80%, or by at least about 90% of the concentration decrease in total sugars.
25. The use according to any of paras 22 to 24, or the Bifidobacterium lactis supernatant for use according to any of paras 22 to 24, wherein compared to Bifidobacterium lactis culture media the concentration of total amino acids has decreased by at least about 0.1 wt %, by at least about 0.2 wt %, or by at least about 0.3 wt %.
26. The use according to any of paras 1 or 4 to 25, or the Bifidobacterium lactis supernatant for use according to any of paras 2 to 25, wherein before pasteurisation the Bifidobacterium lactis supernatant has a viable cell count of from about 1×10⁷ to about 1×10⁹ cfu/ml.
27. The use according to any of paras 1 or 4 to 26, or the Bifidobacterium lactis supernatant for use according to any of paras 2 to 26, wherein the Bifidobacterium lactis supernatant comprises about 4 wt % or less, about 3 wt % or less, about 2 wt % or less, about 1 wt % or less, or about 0.5 wt % or less total sugars.
28. The use according to any of paras 1 or 4 to 27, or the Bifidobacterium lactis supernatant for use according to any of paras 2 to 27, wherein the Bifidobacterium lactis supernatant comprises about 0.5 wt % or more, about 1 wt % or more, about 1.5 wt % or more, or about 2 wt % or more total acids.
29. The use according to any of paras 1 or 4 to 28, or the Bifidobacterium lactis supernatant for use according to any of paras 2 to 28, wherein the Bifidobacterium lactis supernatant comprises about 3.5 wt % or less, about 2 wt % or less, about 1 wt % or less, about 0.8 wt % or less, or about 0.6 wt % or less total amino acids.
30. The use according to any of paras 1 or 4 to 29, or the Bifidobacterium lactis supernatant for use according to any of paras 2 to 29, wherein the Bifidobacterium lactis supernatant has a pH of from about 5 to about 7, from about 5.5. to about 6.5, or of about 6, preferably wherein the Bifidobacterium lactis supernatant has a pH of about 6.2.
31. The use according to any of paras 1 or 4 to 30, or the Bifidobacterium lactis supernatant for use according to any of paras 2 to 30, wherein the Bifidobacterium lactis supernatant is used in combination with one or more probiotics, prebiotics, or synbiotics, preferably wherein the Bifidobacterium lactis supernatant is used in combination with one or more probiotics.
32. The use according to para 31, or the Bifidobacterium lactis supernatant for use according to para 31, wherein the Bifidobacterium lactis supernatant is used in combination with a Bifidobacterium lactis probiotic.
33. The use according to para 32, or the Bifidobacterium lactis supernatant for use according to para 32, wherein the probiotic Bifidobacterium lactis is the same as the Bifidobacterium lactis from which the supernatant is derived.
34. The use according to any of paras 1 or 4 to 33, or the Bifidobacterium lactis supernatant for use according to any of paras 2 to 33, wherein the Bifidobacterium lactis supernatant enhances the growth of one or more bifidobacterial phylogenetic groups selected from: the B. adolescentis group, the B. bifidum group, the B. longum group, and the B. pseudolongum group.
35. The use according to any of paras 1 or 4 to 34, or the Bifidobacterium lactis supernatant for use according to any of paras 2 to 34, wherein the Bifidobacterium lactis supernatant enhances the growth of one or more bifidobacterial species selected from: B. longum, B. animalis, B. adolescentis, B. bifidum, B. catenulatum, B. pseudocatenulatum, B. breve, B. pseudolongum, B. gallicum, B. angulatum, and B. faecale.
36. The use according to any of paras 1 or 4 to 35, or the Bifidobacterium lactis supernatant for use according to any of paras 2 to 35, wherein the subject is a mammal, preferably wherein the subject is a human, a dog, a cat, a rodent, or a rabbit, more preferably wherein the subject is a human.
37. The Bifidobacterium lactis supernatant for use according to any of paras 2 to 36, wherein the subject has or is at risk of a disorder associated with decreased numbers of bifidobacteria in the gut, such as a gastrointestinal disease, obesity, an allergic disease, or regressive autism.
38. The Bifidobacterium lactis supernatant for use according to any of paras 2 to 37, wherein the subject has or is at risk of a gastrointestinal disease.
39. The Bifidobacterium lactis supernatant for use according to any of paras 2 to 38, wherein the subject has a low abundance of bifidobacteria in their gastrointestinal tract and/or faeces, preferably wherein the subject has a low abundance of bifidobacteria in their gastrointestinal tract and/or faeces relative to other microbiota.
40. The Bifidobacterium lactis supernatant for use according to any of paras 2 to 39, wherein the gastrointestinal disease is a gastric disease or an intestinal disease, preferably an intestinal disease.
41. The Bifidobacterium lactis supernatant for use according to any of paras 2 to 40, wherein the gastrointestinal disease is selected from: antibiotic-associated diarrhea, Helicobacter pylori infection, an inflammatory bowel disease (IBD), irritable bowel syndrome (IBS), lactose intolerance, infectious diarrhea, colorectal cancer, chemotherapy-induced diarrhea, and necrotizing enterocolitis.
42. The Bifidobacterium lactis supernatant for use according to any of paras 2 to 41, wherein the gastrointestinal disease is selected from: antibiotic-associated diarrhea, Helicobacter pylori infection, an inflammatory bowel disease (IBD), and irritable bowel syndrome (IBS).
43. The Bifidobacterium lactis supernatant for use according to any of paras 41 or 42, wherein the IBD is Crohn's disease (CD), ulcerative colitis (UC), or pouchitis.
44. A Bifidobacterium lactis supernatant, wherein the Bifidobacterium lactis supernatant has been spray-dried.
45. The Bifidobacterium lactis supernatant according to para 44, wherein the Bifidobacterium lactis supernatant is as defined according to any of paras 4 to 15 or 17 to 30.
45. A supplement comprising a Bifidobacterium lactis supernatant, preferably wherein the supplement is in the form of a capsule or a tablet.
46. A nutritional composition comprising a Bifidobacterium lactis supernatant.
47. The supplement according to para 45, or the nutritional composition according to para 46, wherein the Bifidobacterium lactis supernatant is as defined according to any of paras 4 to 19 or 22 to 30.
48. The supplement according to para 45 or 47, or the nutritional composition according to para 46 or 47, wherein the supplement or nutritional composition comprises one or more probiotics, prebiotics, or synbiotics, preferably wherein the supplement or nutritional composition comprises one or more probiotics.
49. The supplement according to any of paras 45, 47 or 48, or the nutritional composition according to any of paras 46 to 48, wherein the supplement or nutritional composition comprises a Bifidobacterium lactis probiotic, preferably wherein the probiotic Bifidobacterium lactis is the same as the Bifidobacterium lactis from which the supernatant is derived.
50. A method of manufacturing a Bifidobacterium lactis supernatant, the method comprising:
   (a) culturing a Bifidobacterium lactis in a culture media to provide a Bifidobacterium lactis fermentate;
   (b) removing substantially all the Bifidobacterium lactis cells from the Bifidobacterium lactis fermentate to provide a Bifidobacterium lactis supernatant; and
   (c) optionally, pasteurising the Bifidobacterium lactis supernatant.

51. The method according to para 50, wherein the method further comprises a step (d) of drying the Bifidobacterium lactis supernatant, preferably wherein the Bifidobacterium lactis supernatant is dried by spray-drying.
52. The method according to para 50 or 51, wherein the Bifidobacterium lactis supernatant is a Bifidobacterium lactis supernatant according to para 44 or 45.
53. A method of manufacturing a supplement comprising a Bifidobacterium lactis supernatant, the method comprising:
   (a) culturing a Bifidobacterium lactis in a culture media to provide a Bifidobacterium lactis fermentate;
   (b) removing substantially all the Bifidobacterium lactis cells from the Bifidobacterium lactis fermentate to provide a Bifidobacterium lactis supernatant;
   (c) optionally, pasteurising the Bifidobacterium lactis supernatant;
   (d) drying the Bifidobacterium lactis supernatant to provide a Bifidobacterium lactis supernatant powder, optionally wherein the Bifidobacterium lactis supernatant is dried by spray-drying.; and
   (e) encapsulating, compressing and/or packaging the Bifidobacterium lactis supernatant powder to provide a supplement comprising the Bifidobacterium lactis supernatant.
54. The method according to para 53, wherein the Bifidobacterium lactis supernatant is dried by spray-drying.

1-2. (canceled)

3. A method for treating or preventing a gastrointestinal disease by enhancing the growth of bifidobacteria in the gastrointestinal tract of a subject in need of same comprising administering a Bifidobacterium lactis supernatant to the subject.

4. The method according to claim 3, wherein the Bifidobacterium lactis is selected from the group consisting of: Bifidobacterium animalis subsp. lactis CNCM I-3446, Bifidobacterium animalis subsp. lactis B112, Bifidobacterium animalis subsp. lactis BLC1, Bifidobacterium animalis subsp. lactis DSM10140, Bifidobacterium animalis subsp. lactis V9, Bifidobacterium animalis subsp. lactis B1-04, Bifidobacterium animalis subsp. lactis Bi-07, Bifidobacterium animalis subsp. lactis B420, Bifidobacterium animalis subsp. lactis BB-12, Bifidobacterium animalis subsp. lactis AD011, Bifidobacterium animalis subsp. lactis HN019, Bifidobacterium animalis subsp. lactis DN-173 010, Bifidobacterium animalis subsp. lactis ATCC 27536, and Bifidobacterium animalis subsp. lactis VTT E-012010.

5. The method according to claim 3, wherein the Bifidobacterium lactis supernatant is obtained or obtainable by culturing Bifidobacterium lactis in a culture media comprising sugar and yeast extract.

6. The method according to claim 3, wherein the Bifidobacterium lactis supernatant is obtained or obtainable by culturing the Bifidobacterium lactis until stationary phase is reached and/or under anaerobic conditions.

7. The method according to claim 3, wherein the Bifidobacterium lactis supernatant is pasteurised and/or dried.

8. The method according to claim 3, wherein the Bifidobacterium lactis supernatant is administered orally.

9. The method according to claim 3, wherein the Bifidobacterium lactis supernatant is in the form of a supplement or a nutritional composition.

10. The method according to claim 3, wherein compared to Bifidobacterium lactis culture media the Bifidobacterium lactis supernatant has:
(i) a decreased concentration of total sugar;
(ii) an increased concentration of total acids; and
(iii) a decreased concentration of total amino acids.

11. The method according to claim 3, wherein the Bifidobacterium lactis supernatant comprises: (i) about 4 wt % or less total sugars; (ii) about 0.5 wt % or more total acids; and (iii) about 3.5 wt % or less total amino acids.

12. The method according to claim 3, wherein the Bifidobacterium lactis supernatant is used in combination with one or more probiotics, prebiotics, or synbiotics.

13. The method according to claim 3, wherein the subject has or is at risk of a gastrointestinal disease and/or the subject has a low abundance of bifidobacteria in their gastrointestinal tract and/or faeces.

14. The method according to claim 3, wherein the gastrointestinal disease is selected from the group consisting of: antibiotic-associated diarrhea, Helicobacter pylori infection, an inflammatory bowel disease (IBD), irritable bowel syndrome (IBS), lactose intolerance, infectious diarrhea, colorectal cancer, chemotherapy-induced diarrhea, and necrotizing enterocolitis.

15-16. (canceled)
